(19) United States
(12) Patent Application Publication
(10) Pub. No.: US 2026/0055060 A1
(43) Pub. Date: Feb. 26, 2026

(54) USE OF (ISOINDOLIN-2-YL)(4-HYDROXY-3-(ISOINDOLINE-2-CARBONYL)PHENYL)METHANONES IN THE TREATMENT OF TUMOR NECROSIS FACTOR RECEPTOR-ASSOCIATED PROTEIN 1 DYSFUNCTION

(71) Applicants: UNIVERSITY OF NOTRE DAME DU LAC, South Bend, IN (US); UNIVERSITY OF KANSAS, Lawrence, KS (US)

(72) Inventors: Vince Crowley, Lawrence, KS (US); Brian Blagg, South Bend, IN (US); Taylor Merfeld, South Bend, IN (US); Katherine Byrd, South Bend, IN (US); Matthew Rhodes, South Bend, IN (US); Andrew Gutierrez, South Bend, IN (US); Bradley M. Keegan, South Bend, IN (US); Christopher M. Brackett, South Bend, IN (US)

(21) Appl. No.: 19/104,651

(22) PCT Filed: Aug. 21, 2023
(86) PCT No.: PCT/US2023/072590

§ 371 Date: Feb. 18, 2025

Related U.S. Application Data
(60) Provisional application No. 63/373,062, filed on Aug. 21, 2022.

(51) Int. Cl. C07D 209/44; A61K 31/4035; A61K 31/675; A61P 35/00; C07F 9/572

(52) U.S. Cl. CPC C07D 209/44; A61K 31/4035; A61K 31/675; A61P 35/00; C07F 9/5728

(57) ABSTRACT

Described are compounds, compositions, and methods for treating Tumor Necrosis Factor Receptor-Associated Protein 1 (TRAP 1) related diseases and/or disorders, such as cancer.

### CROSS-REFERENCE TO RELATED APPLICATION(S)

This application claims priority to U.S. Provisional Patent Application No. 63/373,062, filed on Aug. 21, 2022, the entire contents of which are fully incorporated herein by reference.

### STATEMENT OF GOVERNMENT INTEREST

This invention was made with government support under Grant numbers CA167079 and CA219907 awarded by The National Institutes of Health, under Grant numbers OKLO3159 and OKLO3060 awarded by the Oklahoma Agricultural Experiment Station, and under Grant P20GM103640 award by the National Institute of General Medical Sciences. The government has certain rights in the invention.

### TECHNICAL FIELD

The present disclosure relates to compounds, compositions, and methods for treating Tumor Necrosis Factor Receptor-Associated Protein 1 (TRAP1) related diseases and/or disorders, such as cancer.

### INTRODUCTION

Tumor Necrosis Factor Receptor-Associated Protein 1 (TRAP1), which belongs to the Hsp90 (heat shock protein 90) family, is a mitochondrial molecular chaperone that helps other proteins fold correctly. TRAP1 is rarely detected in normal cells, but is overexpressed in various cancers such as breast, prostate, pancreatic, lung and colon cancers, and reprograms cellular metabolism to allow cancer cells to adapt to the harsh tumor environment. Since inactivation of TRAP1 induces massive apoptosis in cancer cells in vitro and in vivo, inhibitors targeting TRAP1 are being developed as well as mitochondria-targeted TRAP1 inhibitors.

### SUMMARY

In some aspects, the present disclosure provides compounds of formula (I), or a pharmaceutically acceptable salt thereof,

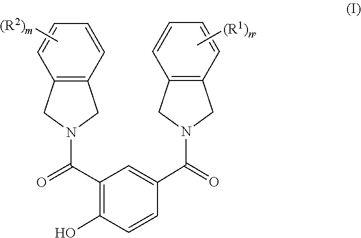

wherein:
R¹, at each occurrence, is independently C₁₋₆alkyl, C₁₋₄haloalkyl, halogen, cyano, —N(R^{1a}) —OR^{1b}, —SR^{1b}, —C(O)R^{1b}, —CO₂R^{1b}, —C(O)N(R^{1a})₂, —SO₂R^{1a}, -L¹-Y¹, —O-L¹-Y¹, —S—L¹-Y¹, —N(R^{1a})-L¹-Y¹, G¹, or —OG¹;
R^{1a}, at each occurrence, is independently hydrogen, C₁₋₄alkyl, or —C(O)C₁₋₄alkyl;
R^{1b}, at each occurrence, is independently hydrogen, C₁₋₄alkyl, C₁₋₂haloalkyl, or —C(O)C₁₋₄alkyl;
L¹, at each occurrence, is independently C₁₋₆alkylene, C₂₋₆alkenylene, or C₂₋₆alkynylene;
Y¹, at each occurrence, is independently hydrogen, cyano, halogen, haloalkyl, —OH, —N(R^{1a})₂, —OR^{1b}, —SR^{1b}, —C(O)R^{1b}, —CO₂R^{1b}, —C(O)N(R^{1a})₂, —SO₂R^{1a},

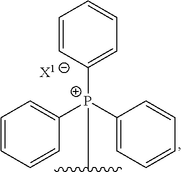

G¹, or —OG¹;
X¹ ⊖ is a pharmaceutically acceptable anion;
G¹ is C₃₋₆cycloalkyl or phenyl, wherein G¹ is optionally substituted with 1-4 substituents selected from the group consisting of C₁₋₄alkyl, C₁₋₂haloalkyl, halogen, cyano, —OC₁₋₄alkyl, and —OC₁₋₂haloalkyl;
R², at each occurrence, is independently C₁₋₆alkyl, C₁₋₄haloalkyl, halogen, cyano, —N(R^{2a})₂, —OR^{2b}, —SR^{2b}, —C(O)R^{2b}, —CO₂R^{2b}, —C(O)N(R^{2a})₂, —SO₂R^{2a}, -L²-Y², —O-L²-Y², —S-L²-Y², —N(R^{2a})-L²_Y², G², or —OG₂;
R^{2a}, at each occurrence, is independently hydrogen, C₁₋₄alkyl, or —C(O)C₁₋₄alkyl;
R^{2b}, at each occurrence, is independently hydrogen, C₁₋₄alkyl, C₁₋₂haloalkyl, or —C(O)C₁₋₄alkyl;
L², at each occurrence, is independently C₁₋₆alkylene, C₂₋₆alkenylene, or C₂₋₆alkynylene;
Y² at each occurrence, is independently hydrogen, cyano, halogen, haloalkyl, —OH, —N(R^{2a})₂, —OR^{2b}, —SR^{2b}, —C(O)R^{2b}, —CO₂R^{2b}, —C(O)N(R^{2a})₂, —SO₂R^{2a},

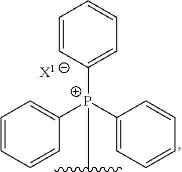

G², or —OG²;
X² ⊕ is a pharmaceutically acceptable anion;
G² is C₃₋₆cycloalkyl or phenyl, wherein G² is optionally substituted with 1-4 substituents selected from the group consisting of C₁₋₄alkyl, C₁₋₂haloalkyl, halogen, cyano, —OC₁₋₄alkyl, and —OC₁₋₂haloalkyl;
n is 0, 1, 2, 3, or 4; and
n is 0, 1, 2, 3, or 4.

In other aspects, the present disclosure provides methods for treating a disease or disorder associated with Tumor Necrosis Factor Receptor-Associated Protein 1 (TRAP-1) dysfunction comprising administering to a subject in need thereof, a therapeutically effective amount of a compound of formula (I), a pharmaceutically acceptable salt thereof, or the pharmaceutical composition.

In other aspects, the present disclosure provides methods for treating a disease or disorder that is cancer.

In other aspects, the present disclosure provides compounds of formula (I), or pharmaceutically acceptable salts or compositions thereof.

### BRIEF DESCRIPTION OF THE DRAWINGS

FIG. 1A shows a Western blot of HeLa cells treated for 6 h with DMSO (control), gamitrinib, MitoQ, compound 35, or compound 36.

FIG. 1B shows a Western blot for 22Rv1 cells treated for 6 h with DMSO (control), gamitrinib, MitoQ, compound 35, or compound 36.

FIG. 1C shows a Western blot displaying a dose-response degradation of NDUFS1, Glutaminase-1, and Sirt3 by compound 35 in HeLa cells.

FIG. 1D shows a Western blot displaying a dose-response degradation of Glutaminase-1 by compound 35 in 22Rv1 cells.

FIG. 1E shows a Western blot displaying a dose-response degradation of NDUFS1, Glutaminase-1, and Sirt3 by compound 36 in HeLa cells

FIG. 1F. shows a Western blot displaying a dose-response degradation of Glutaminase-1 by compound 36 in 22Rv1 cells.

FIG. 2A graphically shows the oxygen consumption rate of HeLa cells treated for 6 h with DMSO (control), gamitrinib, MitoQ, compound 32, compound 35, or compound 36 and measured by Agilent Seahorse XFe assay. High concentrations (25 μM and 50 μM) of compounds 35 and 36 exhibit a statistically significant reduction of oxygen consumption. Displayed results represent the mean+/−SD; Lines above each graph represent statistical comparisons: *P<0.05, **P<0.01, ***P<0.001 by ANOVA, n=5.

FIG. 2B graphically shows oxygen consumption rate of 22Rv1 cells treated for 6 h with DMSO (control), gamitrinib, MitoQ, compound 32, compound 35, or compound 36 and measured by Agilent Seahorse XFe assay. High concentrations (25 μM and 50 μM) of compounds 35 and 36 exhibit a statistically significant reduction of oxygen consumption. Displayed results represent the mean+/−SD; *P<0.05, **P<0.01, ***P<0.001 by ANOVA, n=5.

FIG. 3A graphically shows the results obtained after HeLa cells were stained with tetramethylrhodamine (TMRM) following 6 h treatment with DMSO (control), gamitrinib, MitoQ, compound 32, compound 35, or compounds 36. Quantification of the fluorescence intensity of TMRM measure by Cytatsion5 instrument revealed that high concentrations (25 μM and 50 μM) of compounds 35 and 36 disrupt mitochondrial membrane potential. Graphical representation of the data show the mean+/−SD; *P<0.05, **P<0.01, ***P<0.001 by ANOVA, n=5.

FIG. 3B shows microscopic images taken by a Cystation5 after HeLa cells were stained with TMRM after treatment with DMSO (control), 5 μM MitoQ, 10 μM gamitinib, 50 μM compound 32, 50 μM compound 35, and 50 μM compound 36.

FIG. 4A graphically shows the effect of DMSO (control), gamitrinib, MitoQ, compound 32, compound 35, or compound 36 on mitochondrial proton leak and maximal respiration in HeLa cells. Graphical representation of data shows the mean+/−SD; *P<0.05, **P<0.01, ***P<0.001 by ANOVA, n=5.

FIG. 4B graphically shows the effect of DMSO (control), gamitrinib, MitoQ, compound 32, compound 35, or compound 36 on non-mitochondrial oxygen consumption and spare respiratory capacity in HeLa cells. Graphical representation of data shows the mean+/−SD; *P<0.05, **P<0.01, ***P<0.001 by ANOVA, n=5.

FIG. 4C. graphically shows the effect of DMSO (control), gamitrinib, MitoQ, compound 32, compound 35, or compound 36 on mitochondrial proton leak and maximal respiration in 22Rv1 cells. Graphical representation of data shows the mean+/−SD; *P<0.05, **P<0.01, ***P<0.001 by ANOVA, n=5.

FIG. 4D graphically shows the effect of DMSO (control), gamitrinib, MitoQ, compound 32, compound 35, or compound 36 on non-mitochondrial oxygen consumption and spare respiratory capacity in HeLa cells. Graphical representation of data shows the mean+/−SD; *P<0.05, **P<0.01, ***P<0.001 by ANOVA, n=5.

FIG. 5A graphically shows a comparison of the basal glycolysis Adenosine triphosphate (ATP) production rates and the basal mitochondrial ATP production rates in response to DMSO (control), gamitinib, MitoQ, compound 32, compound 35, or compound 36 in HeLa cells.

FIG. 5B graphically shows the ATP production rates from glycolysis in HeLa cells after initial treatment with TRAP1 inhibitor and subsequent treatment with oligomycin and rotenone/antimycin A.

FIG. 5C graphically shows the ATP production rates from mitochondrial ATP synthesis in HeLa cells after initial treatment with TRAP1 inhibitor and subsequent treatment with oligomycin and rotenone/antimycin A.

FIG. 6A graphically shows the basal oxygen consumption rate measured by Agilent Seahorse XFe in HeLa cells incubated overnight with 10 mM glucose and subsequently treated for 2 h with DMSO (control) followed by BPTES (glutaminase inhibitor), UK5099 (mitochondrial pyruvate transporter inhibitor), and etomoxir (carnitine palmitoyltransferase-1/CPT-1 inhibitor).

FIG. 6B graphically shows the basal oxygen consumption rate measured by Agilent Seahorse XFe in HeLa cells incubated overnight with 2 mM glutamine and subsequently treated for 2 h with DMSO (control) followed by BPTES (glutaminase inhibitor), UK5099 (mitochondrial pyruvate transporter inhibitor), and etomoxir (carnitine palmitoyltransferase-1/CPT-1 inhibitor).

FIG. 6C graphically shows the basal oxygen consumption rate measured by Agilent Seahorse XFe in HeLa cells incubated overnight with 1 mM pyruvate and subsequently treated for 2 h with DMSO (control) followed by BPTES (glutaminase inhibitor), UK5099 (mitochondrial pyruvate transporter inhibitor), and etomoxir (carnitine palmitoyltransferase-1/CPT-1 inhibitor).

FIG. 6D graphically shows the basal oxygen consumption rate measured by Agilent Seahorse XFe in HeLa cells incubated overnight with 10 mM glucose and subsequently treated for 2 h with 50 μM compound 35 followed by BPTES (glutaminase inhibitor), UK5099 (mitochondrial pyruvate transporter inhibitor), and etomoxir (carnitine palmitoyltransferase-1/CPT-1 inhibitor).

FIG. 6E graphically shows the basal oxygen consumption rate measured by Agilent Seahorse XFe in HeLa cells incubated overnight with 2 mM glutamine and subsequently treated for 2 h with 50 μM compound 35 followed by BPTES (glutaminase inhibitor), UK5099 (mitochondrial pyruvate transporter inhibitor), and etomoxir (carnitine palmitoyltransferase-1/CPT-1 inhibitor).

FIG. 6F graphically shows the basal oxygen consumption rate measured by Agilent Seahorse XFe in HeLa cells incubated overnight with 1 mM pyruvate and subsequently treated for 2 h with 50 μM compound 35 followed by BPTES (glutaminase inhibitor), UK5099 (mitochondrial pyruvate transporter inhibitor), and etomoxir (carnitine palmitoyltransferase-1/CPT-1 inhibitor).

FIG. 7 shows a Western blot of HeLa cells treated for 2 h with DMSO (control), gamitrinib, MitoQ, or various concentrations of compound 35.

FIG. 8 illustrates compound 27 interacting with the TRAP1 N-terminal pocket.

FIG. 9A illustrates compound 20 bound to the ATP-binding site of the human TRAP1 receptor and highlights hydrogen-bonding to helix 2 and 4.

FIG. 9B illustrates compound 27 bound to the ATP-binding site of the human TRAPI receptor.

FIG. 10 shows secondary structure assignments for the co-crystal structures of compounds 5, 20, and 27 bound to the human TRAP1 receptor.

FIG. 11 is a model of the hydrophobic binding pocket of human TRAP1 that accommodates methoxy substitution of the (isoindolin-2-yl)(4-hydroxy-3-(isoindoline-2-carbonyl)phenyl)methanone scaffold.

FIG. 12 is an overlay of the crystal structures in complex with compound 34 (green) and 20 (yellow).

FIG. 13A illustrates a symmetrical co-crystal structure of two TRAP1 NM molecules (cyan and green) bound to compound 20 (magenta).

FIG. 13B illustrates a potential allosteric binding site for compound 20.

FIG. 13C is an electrostatic surface map of the TRAP1 binding pocket with compound 20.

FIG. 13D is a surface map highlighting key amino acid residues facilitating compound 20 binding with the human TRAP1 receptor.

### DETAILED DESCRIPTIONS

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various way.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein. The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

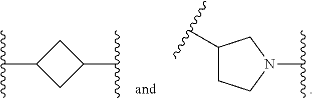 [SMILES: *C1CC(*)C1 ; *C1CCN(*)C1]

Cycloalkylene and heterocyclylene include a geminal divalent groups such as 1,1-C₃₋₆cycloalkylene

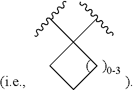

A further example is 1,1-cyclopropylene

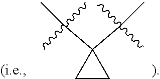 [SMILES: *C1(*)CC1]

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

### 2. Compounds

Compounds of the present disclosure are set forth in the following numbered embodiments. The first embodiment is denoted E1, another embodiment is denoted E2 and so forth.
E1. A compound of formula (I), or a pharmaceutically acceptable salt thereof,

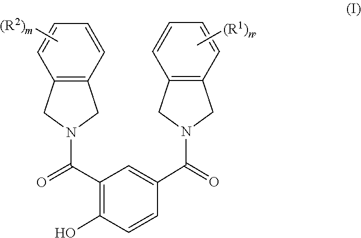

wherein:
R¹, at each occurrence, is independently C₁₋₆alkyl, C₁₋₄haloalkyl, halogen, cyano, —N(R^{1a})₂—OR^{1b}, —SR^{1b}, —C(O)R^{1b}, —CO₂R^{1b}, —C(O)N(R^{1a})₂, —SO₂R^{1a}, -L¹-Y¹, —O-L¹-Y¹, —S-L¹-Y¹, —N(R^{1a})-L¹-Y¹, G¹, or —OG¹;
R^{1a}, at each occurrence, is independently hydrogen, C₁₋₄alkyl, or —C(O)C₁₋₄alkyl;
R^{1b}, at each occurrence, is independently hydrogen, C₁₋₄alkyl, C₁₋₂haloalkyl, or —C(O)C₁₋₄alkyl;
L¹, at each occurrence, is independently C₁₋₆alkylene, C₂₋₆alkenylene, or C₂₋₆alkynylene;
Y¹, at each occurrence, is independently hydrogen, cyano, halogen, haloalkyl, —OH, —N(R^{1a})₂, —OR^{1b}, —SR^{1b}, —C(O)R^{1b}, —CO₂R^{1b}, —C(O)N(R^{1a})₂, —SO₂R^{1a},

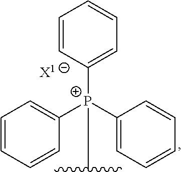

G¹, or —OG¹;
X¹ ⊕ is a pharmaceutically acceptable anion;
G¹ is C₃₋₆cycloalkyl or phenyl, wherein G¹ is optionally substituted with 1-4 substituents selected from the group consisting of C₁₋₄alkyl, C₁₋₂haloalkyl, halogen, cyano, —OC₁₋₄alkyl, and —OC₁₋₂haloalkyl;
R², at each occurrence, is independently C₁₋₆alkyl, C₁₋₄haloalkyl, halogen, cyano, —N(R^{2a})₂, —OR^{2b}, —SR^{2b}, —C(O)R^{2b}, —CO₂R^{2b}, —C(O)N(R^{2a})₂, —SO₂R^{2a}, -L²-Y², —O-L²-Y², —S-L²-Y², —N(R^{2a})-L²-Y², G², or —OG²;
R^{2a}, at each occurrence, is independently hydrogen, C₁₋₄alkyl, or —C(O)C₁₋₄alkyl;
R^{2b}, at each occurrence, is independently hydrogen, C₁₋₄alkyl, C₁₋₂haloalkyl, or —C(O)C₁₋₄alkyl;
L², at each occurrence, is independently C₁₋₆alkylene, C₂₋₆alkenylene, or C₂₋₆alkynylene;
Y² at each occurrence, is independently hydrogen, cyano, halogen, haloalkyl, —OH, —N(R^{2a})₂, —OR^{2b}, —SR^{2b}, —C(O)R^{2b}, —CO₂R^{2b}, —C(O)N(R^{2a})₂, —SO₂R^{2a},

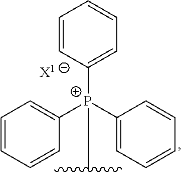

G², or —OG²;
X² ⊕ is a pharmaceutically acceptable anion;
G² is C₃₋₆cycloalkyl or phenyl, wherein G² is optionally substituted with 1-4 substituents selected from the group consisting of C₁₋₄alkyl, C₁₋₂haloalkyl, halogen, cyano, —OC₁₋₄alkyl, and —OC₁₋₂haloalkyl;
n is 0, 1, 2, 3, or 4; and
n is 0, 1, 2, 3, or 4.
E2. The compound of claim 1, or a pharmaceutically acceptable salt thereof, wherein R¹ is halogen, C₁₋₆alkyl, or —OR^{1b}.
E3. The compound of claim 1, or a pharmaceutically acceptable salt thereof, wherein R^{1b} is C₁₋₄alkyl.
E4. The compound of claim 1, or a pharmaceutically acceptable salt thereof, wherein R¹ is fluoro, chloro, bromo, —CH₃, —OCH₃, or —OC₂H₅.
E5. The compound of claim 1, or a pharmaceutically acceptable salt thereof, wherein R² is halogen, —OR^{2b}, or —O-L²-Y².
E6. The compound of claim 1, or a pharmaceutically acceptable salt thereof, wherein R^{2b} is C₁₋₄alkyl.
E7. The compound of claim 1, or a pharmaceutically acceptable salt thereof, wherein L² is C₁₋₆alkylene.
E8. The compound of claim 1, or a pharmaceutically acceptable salt thereof, wherein Y² is —N(R^{2a})₂ or

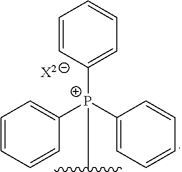

E9. The compound of claim 1, or a pharmaceutically acceptable salt thereof, wherein R^{2a} is C₁₋₄alkyl.
E10. The compound of claim 1, wherein n is 0, 1, or 2.
E11. The compound of claim 1, wherein m is 0, 1, or 2.
E12. The compound of formula 1, or a pharmaceutically acceptable salt thereof, wherein the compound of formula (I) is a compound of formula (I-a):

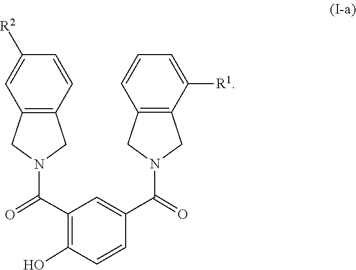

E13. The compound of claim 1, or a pharmaceutically acceptable salt thereof, wherein the compound of formula (I) is a compound of formula (I-b):

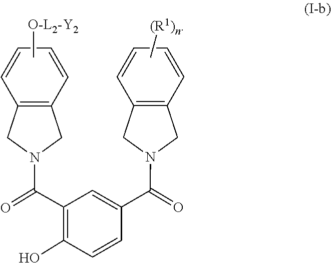

E14. The compound of claim 1, or a pharmaceutically acceptable salt thereof, wherein the compound of formula (I) is a compound of formula (1-ab):

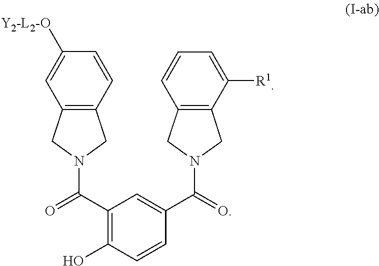

E15. The compound of claim 1, wherein the compound of formula (I) is selected from the group consisting of:

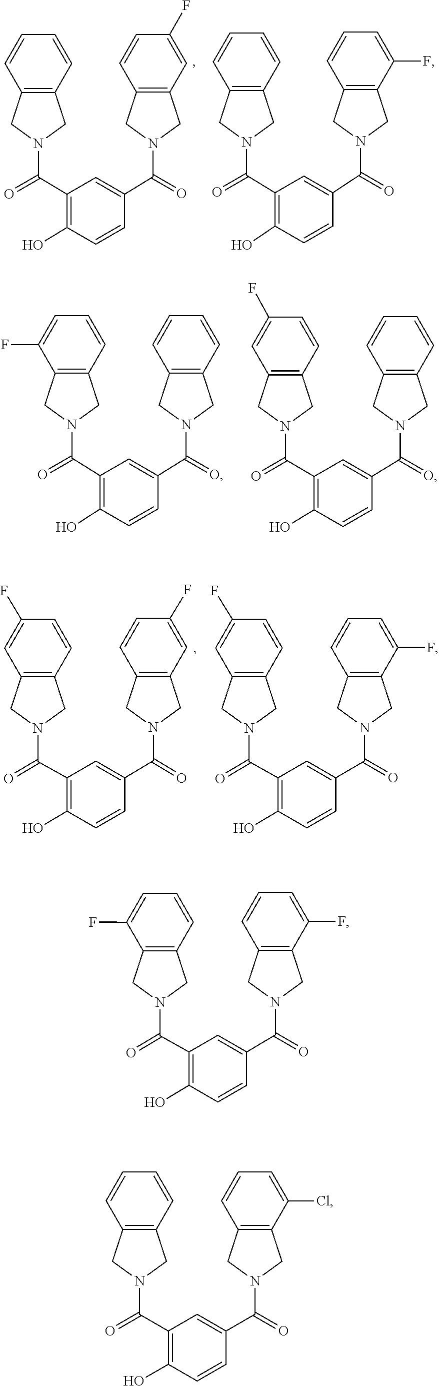

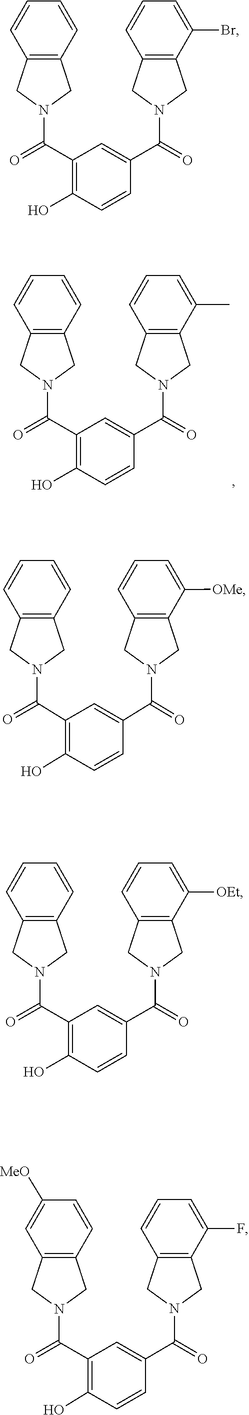 [SMILES: O=C(c1ccc(O)c(C(=O)N2Cc3ccccc3C2)c1)N1Cc2cccc(Br)c2C1 ; Cc1cccc2c1CN(C(=O)c1ccc(O)c(C(=O)N3Cc4ccccc4C3)c1)C2 ; COc1cccc2c1CN(C(=O)c1ccc(O)c(C(=O)N3Cc4ccccc4C3)c1)C2 ; CCOc1cccc2c1CN(C(=O)c1ccc(O)c(C(=O)N3Cc4ccccc4C3)c1)C2 ; COc1ccc2c(c1)CN(C(=O)c1cc(C(=O)N3Cc4cccc(F)c4C3)ccc1O)C2]

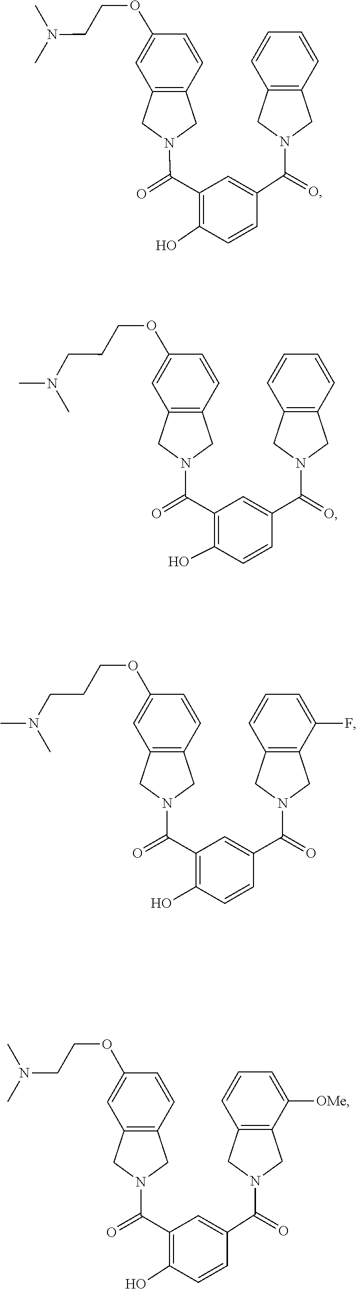 [SMILES: CN(C)CCOc1ccc2c(c1)CN(C(=O)c1cc(C(=O)N3Cc4ccccc4C3)ccc1O)C2 ; CN(C)CCCOc1ccc2c(c1)CN(C(=O)c1cc(C(=O)N3Cc4ccccc4C3)ccc1O)C2 ; CN(C)CCCOc1ccc2c(c1)CN(C(=O)c1cc(C(=O)N3Cc4cccc(F)c4C3)ccc1O)C2 ; COc1cccc2c1CN(C(=O)c1ccc(O)c(C(=O)N3Cc4ccc(OCCN(C)C)cc4C3)c1)C2]

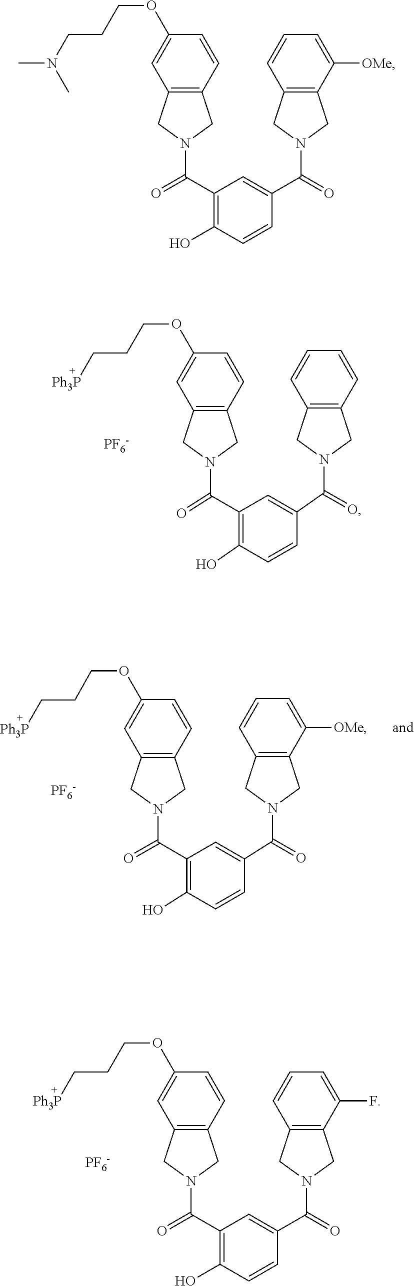

E16. A pharmaceutical composition comprising the compound of claim 1, or a pharmaceutically acceptable salt thereof, and a pharmaceutically acceptable carrier.
E17. A method for treating a disease or disorder associated with Tumor Necrosis Factor Receptor-Associated Protein 1 (TRAP-1) dysfunction comprising administering to a subject in need thereof, a therapeutically effective amount of the compound of claim 1, or a pharmaceutically acceptable salt thereof, or the pharmaceutical composition of claim 16.
E18. The method of claim 17, wherein the disease or disorder is cancer.
E19. A compound of claim 1, or a pharmaceutically acceptable salt thereof, or the pharmaceutical composition of claim 16, for use in the treatment of a disease or disorder associated with Tumor Necrosis Factor Receptor-Associated Protein 1 (TRAP-1) dysfunction.
E20. Use of a compound of claim 1, or a pharmaceutically acceptable salt thereof, or the pharmaceutical composition of claim 16, in the manufacture of a medicament for the treatment of a disease or disorder.

Compound names can be assigned by using Struct=Name naming algorithm as part of CHEMDRAW® ULTRA

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

It should be understood that the compound may possess tautomeric forms, as well as geometric isomers, and that these also constitute an aspect of the invention.

In the compounds of formula (I), and any subformulas, any “hydrogen” or “H,” whether explicitly recited or implicit in the structure, encompasses hydrogen isotopes ¹H (protium) and ²H (deuterium).

### 3. Pharmaceutical Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### 4. General Synthesis

Compounds of formula (I) or any of its subformulas may be synthesized as shown in the following schemes.

Abbreviations which have been used in the Schemes that follow are:
DCM for dichloromethane;
DIPEA is diisopropylethylamine;
EDC is 1-ethyl-3-(3-dimethylaminopropyl)carbodiimide; and
HOBt is hydroxybenzotriazole.

General Scheme 1, below, illustrates the method for preparing compounds of formula (I), described herein.

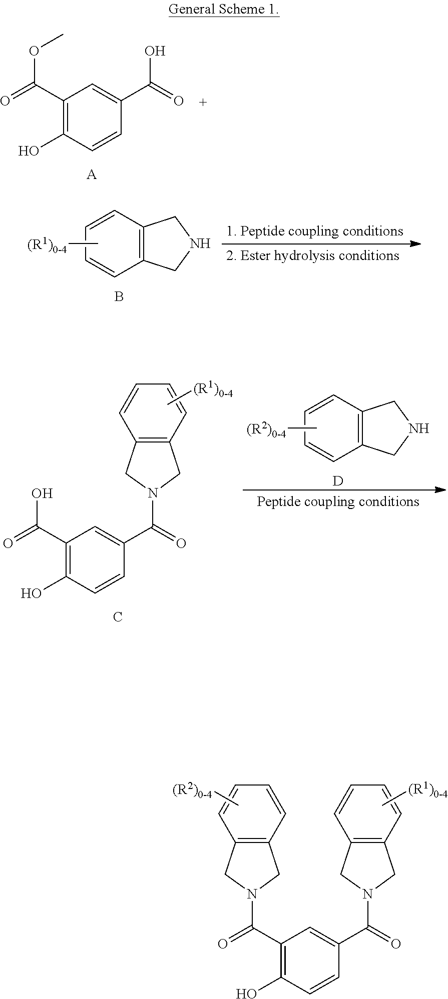

As shown in General Scheme 1 above, compounds of formula (I) may be prepared by coupling 4-hydroxy-3-(methoxycarbonyl)benzoic acid (A) with an isoindoline of formula B under suitable peptide coupling conditions (e.g., in presence of EDC and DIPEA in DCM at room temperature), followed by ester hydrolysis under suitable ester hydrolysis conditions (e.g., in presence of 2M NaOH in 1,4-dioxane at 60° C.) to provide intermediates of formula C. Intermediates of formula C may be coupled with an isoindoline of formula D under suitable peptide coupling conditions (e.g., in presence of EDC and HOBt in DCM at room temperature) to provide compounds of formula (I).

The compounds and intermediates maybe isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above-described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 5. Pharmaceutical Compositions

The disclosed compounds may be incorporated into pharmaceutical compositions suitable for administration to a subject (such as a patient, which may be a human or non-human animal, such as a mammal).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. In general, a suitable dose of the compound is in the range of about 100 μg to about 250 mg per kilogram body weight of the subject per day.

The composition may be administered once, on a continuous basis (e.g. by an intravenous drip), or on a periodic/intermittent basis, including about once per hour, about once per two hours, about once per four hours, about once per eight hours, about once per twelve hours, about once per day, about once per two days, about once per three days, about twice per week, about once per week, and about once per month. The composition may be administered until a desired reduction of symptoms is achieved.

The present compounds, compositions, and methods may be administered as part of a therapeutic regimen along with other treatments appropriate for the particular injury or disease being treated.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed compounds are administered, and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active [e.g., compound of formula (I)] and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound [e.g., a compound of formula (I)], and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

For parenteral administration, the agent can be dissolved or suspended in a physiologically acceptable diluent, such as, e.g., water, buffer, oils with or without solubilizers, surface-active agents, dispersants, or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The pharmaceutical compositions of the present invention may also be administered by nasal aerosol or inhalation through the use of a nebulizer, a dry powder inhaler or a metered dose inhaler. Such compositions are prepared according to techniques well-known in the art of pharmaceutical formulation and may be prepared as solutions in saline, employing benzyl alcohol or other suitable preservatives, absorption promoters to enhance bioavailability, hydrofluorocarbons, and/or other conventional solubilizing or dispersing agents.

Aerosol propellants are required where the pharmaceutical composition is to be delivered as an aerosol under significant pressure. Such propellants include, e.g., acceptable fluorochlorohydrocarbons such as dichlorodifluoromethane, dichlorotetrafluoroethane, and trichloromonofluoromethane; nitrogen; or a volatile hydrocarbon such as butane, propane, isobutane or mixtures thereof.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modem Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

### 6. Tumor Necrosis Factor Receptor-Associated Protein 1 (TRAP1)

The Hsp90 family is distinguished by four different paralogs including Hsp90a, Hsp90p, Grp94 and TRAP1 (Tumor Necrosis Factor Receptor-Associated Protein 1). Hsp90a and Hsp900 reside within the cytosol, while Grp94 and TRAP1 are localized to the endoplasmic reticulum and mitochondria, respectively. Although the Hsp90 family is located and differentially expressed in cells, the Hsp90 family proteins all exist as homodimers. Each protein has three distinct domains: an N-terminal domain (NTD), an intermediate domain (M-domain) and a C-terminal domain (CTD). The CTDs of each protomer on the homodimer interact to give primary dimerization. The M-domain has a large surface capable of interacting with various client proteins. NTD has an ATPase catalytic site where bound ATP is hydrolyzed to ADP. This ATP binding site is also known as the drug binding site. Protomer dimerization is facilitated by the conformational rearrangement of the protein structure resulting from hydrolysis of ATP. A major consequence of NTD conformational change is migration of the ATP-lid, which must close over the ATP-binding site to provide a hydrophobic surface essential for NTD dimerization. For the hinge movement of the ATP-lid, several flexible sequences are placed in the appropriate positions of the ATP-lid. This allows the drug-binding site to adapt to different directed-fits and different inhibitor scaffolds.

Tumor Necrosis Factor Receptor-Associated Protein 1 (TRAP1) maintains mitochondrial integrity and bioenergetics as a member of the 90 kDa heat shock protein (Hsp90) family of molecular chaperones. The Hsp90 chaperones are responsible for the conformational transformation of two-dimensional nascent polypeptides and/or denatured proteins into their biologically active three-dimensional structures. In fact, more than 400 protein substrates are folded by or interact with Hsp90, many of which are associated with pathways that contribute to cancer cell growth and progression. Additionally, TRAP1 is reported to play a role in the metabolic shift from oxidative phosphorylation to glycolysis in cancer cells, thereby playing an essential role in the regulation of mitochondrial metabolism.

During oncogenesis, chaperone function is subverted to maintain homeostasis in the hostile tumor microenvironment, forging a dependency upon Hsp90 and Hsp90 substrates such as Her2, EGFR, and MMP2 amongst others. Consequentially, cancer cells express up to 10-fold higher levels of Hsp90 as compared to normal tissue. Moreover, the upregulated Hsp90 in cancer cells exists predominately in hetero-protein complexes bound to client protein substrates, which exhibit ˜200-fold higher affinity for ATP than the unbound Hsp90 homodimer that resides within normal tissue. Furthermore, Hsp90 ATPase inhibitors are selective over other ATP-dependent proteins due to the presence of a Bergerat fold, which binds ATP in a unique and bent conformation that allows one to target Hsp90 selectively over other ATP binding proteins. In fact, only the GHKL family of proteins bind ATP in similar manner, making the Hsp90 ATP-binding site a druggable target. Hsp90 N-terminal inhibitors inhibit the chaperone's ATPase activity, leading to polyubiquitination and proteasome-mediated degradation of the client proteins, which provides effect similar to a combination therapy where multiple oncogenic pathways are modulated simultaneously.

### 7. Methods of Treatment

Various diseases and disorders, such as cancer, are associated with TRAP1 dysfunction. Cancers associated with TRAP1 dysfunction include, but are not limited to, breast cancer, pancreatic cancer, colon cancer, lung cancer, prostate cancer, cervical cancer, endometrial cancer, glioma, liver cancer, ovarian cancer, brain cancer, bladder cancer, kidney cancer, neuroblastoma, oral cancer, gastric cancer, bone cancer, rectal cancer, thyroid cancer, skin cancer, melanoma, hematologic malignancies, lymphomas, pancreatic tumors, and neuroendocrine tumors.

In addition to cancer, TRAP1 dysfunction has been reported to play a role in diabetes, Alzheimer's disease, Parkinson's disease, thermoregulation, inflammation, and Congenital Anomalies of Kidney and Urinary Tract (CAKUT) disorder.

### EXAMPLES

### Abbreviations

DCM for dichloromethane;
DMF is N,N-dimethylformamide;
DMSO is dimethylsulfoxide;
DIPEA is diisopropylethylamine;
EDC is 1-ethyl-3-(3-dimethylaminopropyl)carbodiimide;
HOBt is hydroxybenzotriazole;
DIAD is diisopropyl azodicarboxylate;
TPP is thiamine pyrophosphate;
TBAI is tetra-n-butylammonium iodide;
eq, eq., or equiv is equivalent(s);
EtOAc is ethyl acetate;
HRMS is high-resolution mass spectrometry.
MeCN is acetonitrile;
MeOH is methanol;
min or min. is minute(s);
h or hr. is hour(s);
mw is microwave irradiation;
Pd(dppf)Cl₂ is [1,1′-Bis(diphenylphosphino)ferrocene]dichloropalladium(II);
PPh₃ is triphenyl phosphine;
rt, RT, or r.t. is room temperature;
sat. is saturated;
TBAI is tetra-n-butylammonium iodide;
TFA is trifluoroacetic acid;
THF is tetrahydrofuran;
OXOPHOS is oxidative phosphorylation;
SAR is structure-activity-relationship;
TPP is triphenylphosphine;
TMRM is tetramethylrhodamine;
FCCP is carbonyl cyanide-4(trifluoromethoxy) phenylhydrazone;
n.d. is not determined;
OCR is oxygen consumption rate;
Rot/AA is rotenone/antimycin A.

### 1. Synthesis of Compounds

### A. Synthesis of Example Intermediates

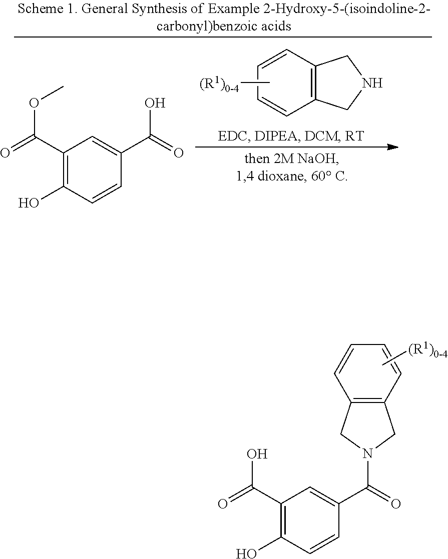

Hydroxy-3-(methoxycarbonyl)benzoic acid (1.27 mmol), HOBt (3.82 mmol), EDC (3.82 mmol) and the corresponding optionally Ri-substituted isoindoline (1.91 mmol) were dissolved in dry dichloromethane (0.2 M). DIPEA (7.65 mmol) was added dropwise to the solution which was stirred at rt under argon for 16 h. The solution was diluted with DCM (5 mL) and washed with water, and the aqueous layer was extracted with DCM 2×. The combined organic layers were washed with 2 N HCl until pH ˜2 and saturated aqueous NaCl solution before isolating. Saturated sodium sulfate was added, and eluent was filtered and concentrated in vacuo. The mixture was purified by column chromatography (hexanes/ethyl acetate 70/30) and yielded colorless amorphic solids (50%-89%).

The corresponding methyl ester (927 μmol) was dissolved in 1,4-dioxane (0.2 M), and 2M NaOH (2.78 mmol) was added. The resulting mixture was heated to 60° C. and stirred for one hour. The mixture was then cooled to room temperature before being diluted with ethyl acetate. The organic layer was washed twice with water, and the combined aqueous layer was acidified to pH ˜2 with 2 N HCl. The cloudy mixture was extracted twice with ethyl acetate, and the combined organic layers were washed with saturated aqueous NaCl solution, dried with sodium sulfate, filtered and the eluent was concentrated in vacuo. The product was used without further purification and yielded colorless amorphic solids (70-95%).

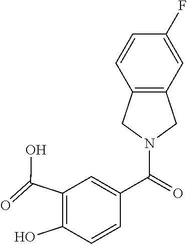 [SMILES: O=C(O)c1cc(C(=O)N2Cc3ccc(F)cc3C2)ccc1O]

5-(5-Fluoroisoindoline-2-carbonyl)-2-hydroxybenzoic acid (7): ¹H NMR (400 MHz, Chloroform-d) δ 8.21-8.15 (m, 2H), 7.72 (dd, J=8.7, 2.2 Hz, 2H), 7.29 (q, J=5.0 Hz, 1H), 7.14 (dd, J=8.5, 5.0 Hz, 1H), 7.05 (d, J=8.5 Hz, 3H), 6.98 (t, J=8.0 Hz, 1H), 6.88 (d, J=8.4 Hz, 1H), 4.99 (d, J=12.0 Hz, 4H), 4.83 (d, J=10.1 Hz, 4H). ¹³C NMR (101 MHz, Chloroform-d) δ 171.67, 169.56, 138.25, 134.56, 131.78, 130.16, 126.69, 123.74, 117.75, 112.33, 110.05, 109.76, 109.53, 55.07, 54.60, 52.69, 52.18, 50.20, 49.98, 49.77, 49.55, 49.34.

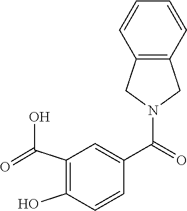 [SMILES: O=C(O)c1cc(C(=O)N2Cc3ccccc3C2)ccc1O]

2-Hydroxy-5-(isoindoline-2-carbonyl)benzoic acid (8): ¹H NMR (400 MHz, CDCl₃) δ 8.18 (d, J=2.3 Hz, 1H), 7.72 (dd, J=8.6, 2.3 Hz, 1H), 7.35-7.26 (m, 2H), 7.17 (d, J=7.2 Hz, 1H), 7.03 (d, J=8.6 Hz, 1H), 5.02 (s, 2H), 4.85 (s, 2H). ¹³C NMR (101 MHz, CDCl₃) δ 171.73, 169.66, 163.28, 136.21, 136.04, 134.50, 130.12, 127.92, 127.61, 126.88, 122.94, 122.44, 117.66, 112.40, 55.19.

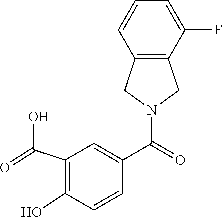 [SMILES: O=C(O)c1cc(C(=O)N2Cc3cccc(F)c3C2)ccc1O]

2-(Benzyloxy)-5-(4-chloroisoindoline-2-carbonyl)benzoic acid (9): ¹H NMR (400 MHz, DMSO-d₆) δ 12.91 (s, 1H), 7.91 (t, J=2.1 Hz, 1H), 7.82 (td, J=11.3, 8.6 Hz, 1H), 7.52 (d, J=7.1 Hz, 2H), 7.45-7.24 (m, 7H), 5.28 (s, 2H), 4.93 (d, J=6.0 Hz, 2H), 4.84 (d, J=6.4 Hz, 2H). ¹³C NMR (101 MHz, DMSO) δ 167.9 and 167.8 (rotamers), 166.8, 158.1, 139.6 and 138.6 (rotamers), 136.7, 135.4 and 134.5 (rotamers), 132.0 and 131.8 (rotamers), 130.1, 129.7 and 129.7 (rotamers), 128.4, 128.1, 128.0, 127.9, 127.8, 127.6, 127.2 and 127.1 (rotamers), 121.8, 113.4, 69.7, 55.1 and 53.7 (rotamers), 53.1 and 51.8 (rotamers). HRMS (ESI) caled for C₂₃H₁₉ClNO₄ [M+H]408.0997, found 408.0987.

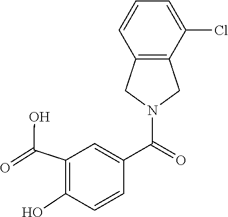 [SMILES: O=C(O)c1cc(C(=O)N2Cc3cccc(Cl)c3C2)ccc1O]

5-(4-Chloroisoindoline-2-carbonyl)-2-hydroxybenzoic acid (10): ¹H NMR (400 MHz, DMSO-d₆) δ 8.06 (d, J=2.3 Hz, 1H), 7.83 (td, J=8.8, 2.3 Hz, 1H), 7.41-7.22 (m, 3H), 7.05 (dd, J=8.6, 2.9 Hz, 1H), 4.93 (d, J=5.2 Hz, 2H), 4.84 (d, J=6.8 Hz, 2H). ¹³C NMR (101 MHz, DMSO) δ 171.4, 167.8 and 167.8 (rotamers), 162.3 and 162.2 (rotamers), 139.6 and 138.7 (rotamers), 135.4 and 134.5 (rotamers), 134.5 and 134.4 (rotamers), 130.0, 129.7 and 129.7 (rotamers), 127.9 and 127.6 (rotamers), 127.3 and 127.2 (rotamers), 127.1 and 127.0 (rotamers), 121.8 and 121.8 (rotamers), 117.1, 113.0 and 112.9 (rotamers), 55.1 and 53.7 (rotamers), 53.1 and 51.8 (rotamers). HRMS (ESI) calc'd for C₁₆H₁₃ClNO₄ [M+H]318.0535, found 318.0528.

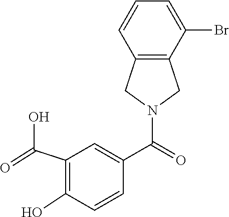 [SMILES: O=C(O)c1cc(C(=O)N2Cc3cccc(Br)c3C2)ccc1O]

5-(4-Bromoisoindoline-2-carbonyl)-2-hydroxybenzoic acid (11): ¹H NMR (400 MHz, DMSO-d₆) δ 8.06 (d, J=2.3 Hz, 1H), 7.83 (ddd, J=10.8, 8.4, 2.3 Hz, 1H), 7.49 (t, J=8.7 Hz, 1H), 7.44-7.20 (m, 2H), 7.04 (dd, J=8.6, 4.5 Hz, 1H), 4.95 (d, J=5.2 Hz, 2H), 4.77 (d, J=7.6 Hz, 2H). ¹³C NMR (101 MHz, DMSO) δ 171.4, 167.8 and 167.7 (rotamers), 162.3 and 162.2 (rotamers), 139.3 and 138.5 (rotamers), 137.4 and 136.5 (rotamers), 134.6 and 134.4 (rotamers), 130.2 and 130.1 (rotamers), 130.0, 129.9 and 129.8 (rotamers), 127.1 and 127.0 (rotamers), 122.3 and 122.2 (rotamers), 117.1, 116.7 and 116.4 (rotamers), 113.0 and 112.9 (rotamers), 55.4 and 55.3 (rotamers), 53.6 and 53.4 (rotamers). HRMS (ESI) calc'd for C₁₆H₁₃BrNO₄ [M+H]362.0022, found 362.0031.

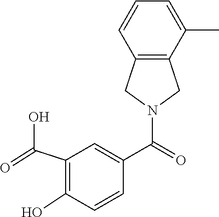 [SMILES: Cc1cccc2c1CN(C(=O)c1ccc(O)c(C(=O)O)c1)C2]

2-Hydroxy-5-(4-methylisoindoline-2-carbonyl)benzoic acid (12): ¹H NMR (400 MHz, DMSO-d₆) δ 8.05 (d, J=2.3 Hz, 1H), 7.83 (ddd, J=8.9, 7.2, 2.3 Hz, 1H), 7.25-6.99 (m, 4H), 4.81 (dd, J=24.1, 12.2 Hz, 4H), 2.20 (d, J=53.2 Hz, 3H, rotamers). ¹³C NMR (101 MHz, DMSO) δ 171.4, 167.8 and 167.8 (rotamers), 162.1, 136.7 and 136.0 (rotamers), 135.7 and 135.1 (rotamers), 134.5 and 134.4 (rotamers), 132.4 and 132.3 (rotamers), 130.0 and 129.9 (rotamers), 128.0 and 128.0 (rotamers), 127.7 and 127.6 (rotamers), 127.5 and 127.4 (rotamers), 120.0 and 120.0 (rotamers), 117.0, 113.0 and 112.8 (rotamers), 54.7 and 53.5 (rotamers), 52.7 and 51.6 (rotamers), 18.3 and 18.2 (rotamers). HRMS (ESI) calc'd for C₁₇H₁₆NO₄ [M+H]298.1030, found 298.1069.

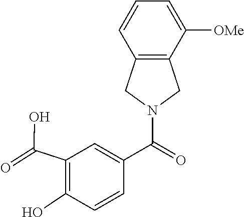 [SMILES: COc1cccc2c1CN(C(=O)c1ccc(O)c(C(=O)O)c1)C2]

2-Hydroxy-5-(4-methoxyisoindoline-2-carbonyl)benzoic acid (13): ¹H NMR (400 MHz, DMSO-d₆) δ 8.05 (d, J=2.2 Hz, 1H), 7.82 (dt, J=8.5, 2.0 Hz, 1H), 7.28 (dt, J=12.2, 7.8 Hz, 1H), 7.04 (dd, J=8.6, 2.7 Hz, 1H), 6.99-6.83 (m, 2H), 4.83 (d, J=10.3 Hz, 2H), 4.72 (d, J=16.4 Hz, 2H), 3.79 (d, J=29.0 Hz, 3H, rotamers). ¹³C NMR (101 MHz, DMSO) δ 171.4, 167.8 and 167.7 (rotamers), 162.2, 154.5 and 154.2 (rotamers), 138.7 and 137.7 (rotamers), 134.5 and 134.5 (rotamers), 129.9 and 129.8 (rotamers), 129.5 and 129.3 (rotamers), 127.3, 124.4 and 123.5 (rotamers), 117.0, 114.9 and 114.8 (rotamers), 112.9, 109.2 and 109.1 (rotamers), 55.3 and 55.2 (rotamers), 54.7 and 50.2 (rotamers), 52.7 and 52.3 (rotamers). HRMS (ESI) calc'd for C₁₇H₁₆NO₅ [M+H]314.1023, found 314.1017.

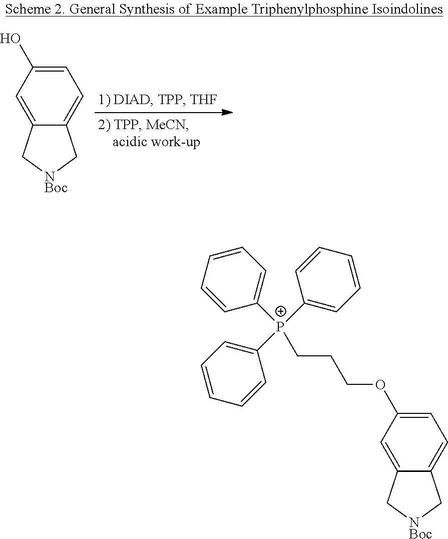 [SMILES: CC(C)(C)OC(=O)N1Cc2ccc(O)cc2C1 ; CC(C)(C)OC(=O)N1Cc2ccc(OCCC[P+](c3ccccc3)(c3ccccc3)c3ccccc3)cc2C1]

To a solution of 5-hydroxy-1,3-dihydro-isoindole-2-carboxylic acid tert-butyl ester (1 eq) in THF (0.2 M) at 0° C. was added 3-bromo-1-propanol (1.3 eq) and triphenylphosphine (1.3 eq). Diisopropyl azodicarboxylate (1.3 eq) was added dropwise. When addition was complete, the reaction mixture was allowed to warm to room temperature and stirred overnight. The volatiles were removed in vacuo and the mixture was purified by column chromatography (hexanes/ethyl acetate 9/1). To a solution of 5-(3-Bromo-propoxy)-1,3-dihydro-isoindole-2-carboxylic acid tert-butyl ester (1 eq) in acetonitrile (0.5 M) was added triphenylphosphine (1.05 eq) and the reaction was refluxed for 16 h. After the reaction was cooled to room temperature, excess triphenylphosphine was removed by extraction with n-hexane (3×). The crude salt was used without further purification. To a solution of crude (3-((2-(tert-butoxycarbonyl)isoindolin-5-yl)oxy)propyl)triphenylphosphonium bromide in CH₂Cl₂ (0.2 M) was added 4 N HCl in 1,4-dioxane (10 eq), and the mixture was stirred overnight. The reaction was then triturated with ether. The suspension was filtered and the resulting solid washed with ether to afford (3-(isoindolin-5-yloxy)propyl)triphenylphosphonium chloride hydrochloride as a beige solid.

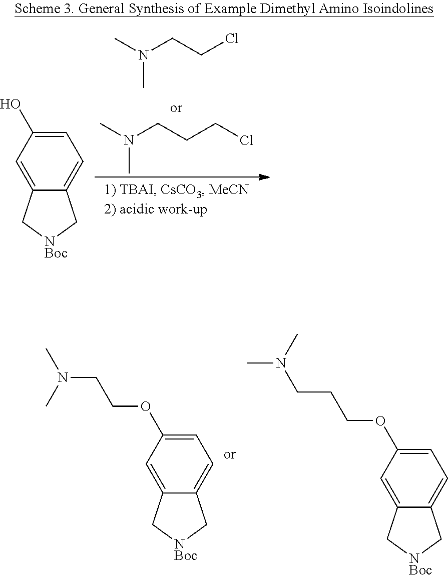

A suspension of tert-butyl 5-hydroxyisoindoline-2-carboxylate (1 eq), 2-chloro-N,N-dimethylethylamine hydrochloride (2.5 eq), cesium carbonate (5 eq), and tetrabutylammonium iodide (0.1 eq) in MeCN (0.2 M) was heated overnight at 90° C. The mixture was cooled to room temperature and diluted with 15% methanol in CH₂Cl₂. The resulting mixture was washed twice with water and once with brine and dried with anhydrous Na₂SO₄ before being concentrated in vacuo and purified by column chromatography (DCM/MeOH 95/5). To a solution of tert-butyl 5-(2-(dimethylamino)propoxy)isoindoline-2-carboxylate in CH₂Cl₂ (0.2 M) was added 4 N HCl in 1,4-dioxane (10 eq), and the mixture was stirred overnight. The reaction was then triturated with ether. The suspension was filtered and the resulting solid washed with ether to afford the products.

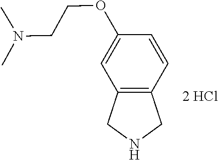

2-(Isoindolin-5-yloxy)-N,N-dimethylethan-1-amine dihydrochloride (36): ¹H NMR (400 MHz, Methanol-d₄) δ 7.63 (dq, J=28.2, 10.4, 9.5 Hz, 1H), 7.42 (dd, J=28.4, 14.4 Hz, 2H), 4.99-4.58 (m, 6H), 4.00-3.79 (m, 2H), 3.27 (dd, J=21.5, 12.0 Hz, 6H). ¹³C NMR (101 MHz, CH₃OH+D₂O) δ 159.95, 137.46, 128.60, 125.13, 117.04, 110.14, 63.62, 57.61, 51.92, 51.47, 49.70, 49.49, 49.27, 49.06, 48.84, 48.63, 48.41, 43.98.

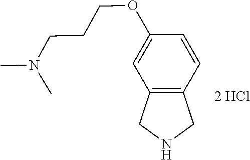 [SMILES: CN(C)CCCOc1ccc2c(c1)CNC2]

3-(Isoindolin-5-yloxy)-N,N-dimethylpropan-1-amine dihydrochloride (37): ¹H NMR (400 MHz, CD₃OD) δ 7.34 (d, J=8.4 Hz, 1H), 7.06 (d, J=2.3 Hz, 1H), 7.00 (dd, J=8.5, 2.3 Hz, 1H), 4.63 (s, 2H), 4.58 (s, 2H), 4.14 (t, J=5.8 Hz, 2H), 3.43-3.33 (m, 2H), 2.96 (s, 6H), 2.27 (dq, J=7.8, 5.8 Hz, 2H). ¹³C NMR (101 MHz, CD₃OD) δ 160.7, 137.2, 127.7, 124.9, 116.8, 109.8, 66.4, 56.4, 51.8, 51.3, 43.6, 25.6. HRMS (ESI) calc'd for C₁₃H₂₁N₂O [M+H]221.1648, found 221.1650.

### B. Synthesis of Example Final Compounds

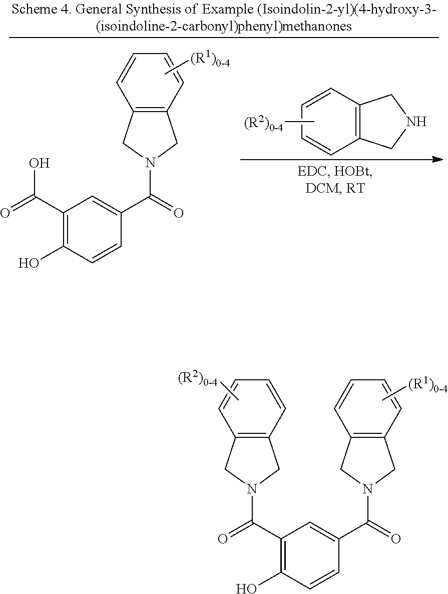

The corresponding optionally R¹-substituted 2-hydroxy-5-(isoindoline-2-carbonyl)benzoic acid (160 μmol), HOBt (479 μmol), EDC (479 mol) and the corresponding optionally R²-substituted isoindoline (239 μmol) were dissolved in dry dichloromethane (0.2 M). DIPEA (958 μmol) was added, and the solution was stirred at rt under argon for 16 h. The solution was taken up in DCM and water, and the aqueous layer was extracted with DCM 2×. The combined organic layers were washed with 2 N HCl, and with saturated aqueous NaCl solution before being dried with saturated sodium sulfate, filtered, and eluent concentrated in vacuo. The mixture was purified by column chromatography (DCM/MeOH 95/5). Work-up yielded colorless amorphic solids 5%-56%.

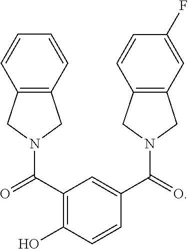 [SMILES: O=C(c1ccc(O)c(C(=O)N2Cc3ccccc3C2)c1)N1Cc2ccc(F)cc2C1]

(4-Fluoroisoindolin-2-yl)(4-hydroxy-3-(isoindoline-2-carbonyl)phenyl)methanone (15): ¹H NMR (400 MHz, Chloroform-d) δ 11.45 (s, 1H), 7.99 (d, J=2.1 Hz, 1H), 7.71-7.63 (m, 1H), 7.32 (t, J=5.1 Hz, 4H), 7.26-6.86 (m, 4H), 5.13 (s, 4H), 5.03 (d, J=11.6 Hz, 2H), 4.87 (d, J=9.9 Hz, 2H). 13C NMR (101 MHz, CDCl₃) δ 136.98, 135.29, 134.44, 134.23, 131.96, 130.69, 130.26, 128.42, 124.30, 123.53, 123.48, 123.18, 121.40, 116.74, 90.47, 77.40, 77.08, 76.77, 53.91, 52.64, 52.16, 50.16, 50.07, 49.86, 49.64, 49.43, 49.21.

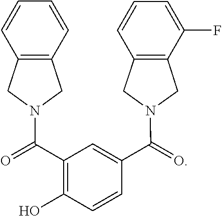 [SMILES: O=C(c1ccc(O)c(C(=O)N2Cc3ccccc3C2)c1)N1Cc2cccc(F)c2C1]

(4-Fluoroisoindolin-2-yl)(4-hydroxy-3-(isoindoline-2-carbonyl)phenyl)methanone (16): ¹H NMR (400 MHz, Chloroform-d) δ 11.42 (s, 1H), 7.97 (s, 1H), 7.67 (d, J=7.8 Hz, 1H), 7.30 (d, J=12.1 Hz, 4H), 7.23 (d, J=6.9 Hz, 1H), 7.17-6.91 (m, 3H), 5.20-5.01 (m, 6H), 4.90 (d, J=12.8 Hz, 2H). ¹³C NMR (101 MHz, CDCl₃) δ 169.68, 169.43, 131.89, 130.24, 128.18, 127.95, 122.62, 118.63, 118.16, 114.19, 77.39, 77.07, 76.75, 55.33, 53.48, 53.07, 52.22, 49.96, 29.72.

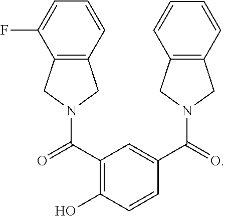 [SMILES: O=C(c1ccc(O)c(C(=O)N2Cc3cccc(F)c3C2)c1)N1Cc2ccccc2C1]

(4-Fluoroisoindolin-2-yl)(4-hydroxy-3-(isoindoline-2-carbonyl)phenyl)methanone (17): ¹H NMR (400 MHz, Chloroform-d) δ 11.27 (s, 1H), 7.96 (d, J=2.1 Hz, 1H), 7.69 (d, J=8.6 Hz, 1H), 7.31 (s, 4H), 7.21 (s, 1H), 7.05 (dd, J=35.0, 8.7 Hz, 3H), 5.16 (s, 4H), 5.05 (s, 2H), 4.90 (s, 2H). ¹³C NMR (101 MHz, Chloroform-d) δ 169.67, 169.08, 161.30, 136.08, 135.99, 131.72, 130.01, 127.73, 127.44, 126.34, 122.74, 122.26, 118.10, 117.65, 116.70, 55.03, 52.62.

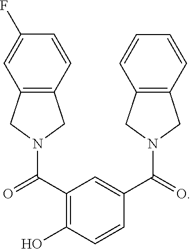 [SMILES: O=C(c1ccc(O)c(C(=O)N2Cc3ccc(F)cc3C2)c1)N1Cc2ccccc2C1]

(5-Fluoroisoindolin-2-yl)(2-hydroxy-5-(isoindoline-2-carbonyl)phenyl)methanone (18): 1H NMR (400 MHz, CDCl3) δ 11.36 (s, 1H), 7.97 (d, J=2.1 Hz, 1H), 7.72-7.63 (m, 1H), 7.42-7.27 (m, 4H), 7.24-7.18 (m, 1H), 7.09 (d, J=8.6 Hz, 1H), 7.01 (d, J=8.6 Hz, 2H), 5.08 (d, J=21.1 Hz, 6H), 4.88 (s, 2H). 13C NMR (126 MHz, CDCl3) δ 169.9, 169.3, 161.9, 136.3, 136.2, 131.9 (2C), 131.8, 128.2, 128.1 (2C), 128.0, 127.9, 127.6, 127.6, 126.5, 123.0, 122.5, 117.8, 117.8 (2C), 116.7, 55.2, 52.8. HRMS (ESI) m/z [M+Na]⁺ for C₂₄H₁₉FN₂O₃Na 425.1277, found 425.1273.

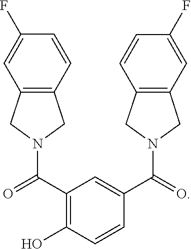 [SMILES: O=C(c1ccc(O)c(C(=O)N2Cc3ccc(F)cc3C2)c1)N1Cc2ccc(F)cc2C1]

(4-Fluoroisoindolin-2-yl)(4-hydroxy-3-(isoindoline-2-carbonyl)phenyl)methanone (19): ¹H NMR (400 MHz, Chloroform-d) δ 7.93 (d, J=2.1 Hz, 1H), 7.64 (dd, J=8.6, 2.1 Hz, 1H), 7.23-7.11 (m, 1H), 7.10-6.84 (m, 5H), 5.19-4.72 (m, 8H). ¹³C NMR (101 MHz, Chloroform-d) δ 169.93, 169.32, 161.94, 133.22, 130.24, 128.10, 123.38, 123.21, 118.63, 118.12, 116.62, 53.06, 52.26, 49.99.

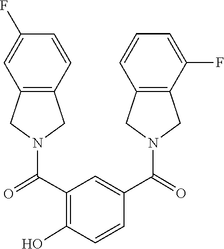 [SMILES: O=C(c1ccc(O)c(C(=O)N2Cc3ccc(F)cc3C2)c1)N1Cc2cccc(F)c2C1]

(4-Fluoroisoindolin-2-yl)(4-hydroxy-3-(isoindoline-2-carbonyl)phenyl)methanone (20): ¹H NMR (400 MHz, Chloroform-d) δ 7.90 (d, J=2.0 Hz, 1H), 7.61 (s, 1H), 7.29-7.22 (m, 1H), 7.18-6.80 (m, 6H), 5.10-4.76 (m, 8H). ¹³C NMR (101 MHz, CDCl₃) δ 169.64, 169.42, 131.91, 128.14, 118.64, 117.85, 114.58, 77.38, 77.27, 77.07, 76.75, 55.31, 53.07, 52.21, 50.03, 49.98, 49.82.

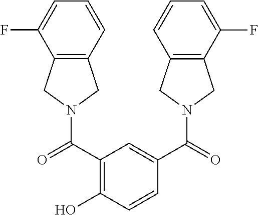 [SMILES: O=C(c1ccc(O)c(C(=O)N2Cc3cccc(F)c3C2)c1)N1Cc2cccc(F)c2C1]

(4-Fluoroisoindolin-2-yl)(4-hydroxy-3-(isoindoline-2-carbonyl)phenyl)methanone (21): ¹H NMR (400 MHz, Chloroform-d) δ 11.30 (s, 1H), 7.96 (d, J=2.1 Hz, 1H), 7.69 (d, J=8.6 Hz, 1H), 7.31 (s, 2H), 7.10 (d, J=8.6 Hz, 2H), 7.00 (t, J=8.6 Hz, 3H), 5.16 (s, 4H), 5.08 (s, 2H), 4.93 (s, 2H). ¹³C NMR (101 MHz, Chloroform-d) δ 169.91, 169.28, 161.94, 132.12, 130.24, 128.10, 123.38, 123.21, 118.63, 118.03, 116.72, 53.09, 52.21, 50.00.

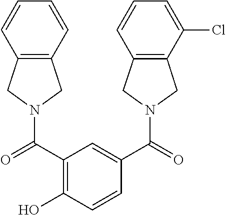 [SMILES: O=C(c1ccc(O)c(C(=O)N2Cc3ccccc3C2)c1)N1Cc2cccc(Cl)c2C1]

(4-Chloroisoindolin-2-yl)(4-hydroxy-3-(isoindoline-2-carbonyl)phenyl)methanone (22): ¹H NMR (400 MHz, CDCl₃) δ 11.49 (s, 1H), 7.99 (d, J=2.1 Hz, 1H), 7.73-7.62 (m, 1H), 7.44 (t, J=8.9 Hz, 1H), 7.30 (d, J=12.2 Hz, 4H), 7.26-7.05 (m, 3H), 5.14 (s, 5H), 5.03 (s, 1H), 4.96 (s, 1H), 4.86 (s, 1H). ¹³C NMR (101 MHz, CDCl₃) δ 169.9 and 169.5 (rotamers), 162.3, 138.3, 138.2, 137.4, 132.2, 131.9, 131.1, 130.7, 130.0, 129.6, 128.4, 128.2, 121.9, 121.4, 118.2, 118.0, 117.4, 116.8, 56.5, 56.2, 54.4, 54.0.

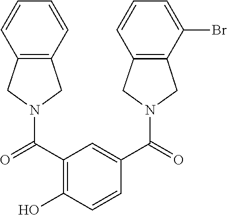 [SMILES: O=C(c1ccc(O)c(C(=O)N2Cc3ccccc3C2)c1)N1Cc2cccc(Br)c2C1]

(4-Bromoisoindolin-2-yl)(4-hydroxy-3-(isoindoline-2-carbonyl)phenyl)methanone (23): (45%, white solid)—¹H NMR (400 MHz, CDCl₃) δ 11.48 (s, 1H), 7.99 (d, J=2.1 Hz, 1H), 7.67 (dd, J=15.8, 8.6 Hz, 1H), 7.44 (t, J=8.8 Hz, 1H), 7.39-7.26 (m, 4H), 7.25-7.05 (m, 3H), 5.13 (s, 5H), 5.02 (s, 1H), 4.96 (s, 1H), 4.85 (s, 1H). ¹³C NMR (101 MHz, CDCl₃) δ 169.9 and 169.5 (rotamers), 162.2 and 162.1 (rotamers), 138.3 and 138.2 (rotamers), 137.3, 136.0, 135.3, 132.2 and 131.9 (rotamers), 131.1 and 130.7 (rotamers), 130.0 and 129.6 (rotamers), 128.3, 128.2, 128.1, 128.0, 126.2 and 126.1 (rotamers), 122.7, 121.9, 121.4, 118.2 and 118.0 (rotamers), 117.4, 117.0 and 116.9 (rotamers), 56.5, 56.2, 54.4, 54.0. HRMS (ESI) calc'd for C₂₄H₂₀BrN₂O₃ [M+H]463.0652, found 463.0644.

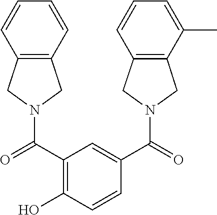 [SMILES: Cc1cccc2c1CN(C(=O)c1ccc(O)c(C(=O)N3Cc4ccccc4C3)c1)C2]

(4-Hydroxy-3-(isoindoline-2-carbonyl)phenyl)(4-methylisoindolin-2-yl)methanone (24): (8%)—¹H NMR (400 MHz, CDCl₃) δ 11.45 (d, J=10.4 Hz, 1H), 7.98 (t, J=2.3 Hz, 1H), 7.67 (dt, J=8.6, 2.6 Hz, 1H), 7.31 (s, 4H), 7.25-6.97 (m, 4H), 5.13 (s, 4H), 5.06 (s, 1H), 4.99 (s, 1H), 4.89 (s, 1H), 4.78 (s, 1H), 2.28 (d, J=47.0 Hz, 3H, rotamers). ¹³C NMR (101 MHz, CDCl₃) δ 170.0 and 169.6 (rotamers), 162.0 and 162.0 (rotamers), 136.2, 135.6, 135.4, 133.2, 132.6, 132.0, 128.7, 128.5, 128.4, 128.2, 128.1, 128.1, 126.7, 126.6, 120.3, 119.8, 118.0, 117.9, 116.9, 55.8, 54.7, 53.3, 52.3, 18.9 and 18.9 (rotamers). HRMS (ESI) calc'd for C₂₅H₂₃N₂O₃ [M+H]399.1703, found 399.1703.

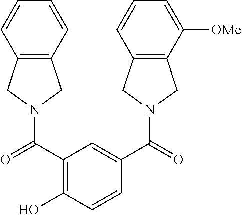 [SMILES: COc1cccc2c1CN(C(=O)c1ccc(O)c(C(=O)N3Cc4ccccc4C3)c1)C2]

(4-Hydroxy-3-(isoindoline-2-carbonyl)phenyl)(4-methoxyisoindolin-2-yl)methanone (25) (16%)—¹H NMR (400 MHz, CDCl₃) δ 11.43 (s, 1H), 7.98 (d, J=2.1 Hz, 1H), 7.67 (dt, J=11.2, 8.5, 2.1 Hz, 1H), 7.37-7.21 (m, SH), 7.08 (dd, J=8.6, 3.5 Hz, 1H), 6.99-6.72 (m, 2H), 5.20-4.95 (m, 6H), 4.83 (d, J=20.6 Hz, 2H), 3.84 (d, J=23.2 Hz, 3H, rotamers). ¹³C NMR (101 MHz, CDCl₃) δ 170.0, 169.5 and 169.4 (rotamers), 161.9 and 161.9 (rotamers), 155.2 and 154.7 (rotamers), 138.2 and 138.1 (rotamers), 132.2 and 131.9 (rotamers), 129.8 and 129.5 (rotamers), 128.2 and 128.2 (rotamers), 128.1, 128.0, 127.9, 127.9, 126.7 and 126.5 (rotamers), 124.7, 123.2, 122.7, 118.0 and 117.9 (rotamers), 117.0 and 116.8 (rotamers), 115.0 and 114.5 (rotamers), 109.2 and 108.8 (rotamers), 55.7, 55.5 and 55.3 (rotamers), 53.6, 53.4 and 53.4 (rotamers), 52.1 and 51.0 (rotamers). HRMS (ESI) calc'd for C₂₅H₂₃N₂O₄ [M+H]415.1652, found 415.1663.

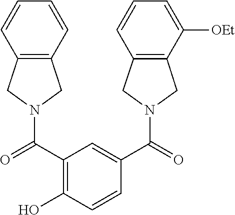 [SMILES: CCOc1cccc2c1CN(C(=O)c1ccc(O)c(C(=O)N3Cc4ccccc4C3)c1)C2]

(4-Ethoxyisoindolin-2-yl)(4-hydroxy-3-(isoindoline-2-carbonyl)phenyl)methanone (26): (25%)—¹H NMR (400 MHz, CDCl₃) δ 11.43 (s, 1H), 7.97 (d, J=2.1 Hz, 1H), 7.67 (td, J=8.9, 2.1 Hz, 1H), 7.38-7.19 (m, 5H), 7.09 (t, J=8.7 Hz, 1H), 6.94-6.71 (m, 2H), 5.24-4.92 (m, 6H), 4.82 (d, J=29.2 Hz, 2H), 4.07 (dq, J=14.0, 7.0 Hz, 2H), 1.40 (dt, J=21.0, 7.0 Hz, 3H). ¹³C NMR (101 MHz, CDCl₃) δ 169.92, 161.81, 154.02, 137.99, 132.00, 129.59, 129.32, 128.02, 126.56, 124.65, 117.90, 114.67, 109.94, 109.44, 77.37, 77.05, 76.73, 63.65, 63.55, 55.70, 53.30, 14.85.

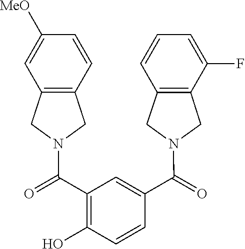 [SMILES: COc1ccc2c(c1)CN(C(=O)c1cc(C(=O)N3Cc4cccc(F)c4C3)ccc1O)C2]

(4-Fluoroisoindolin-2-yl)(4-hydroxy-3-(5-methoxyisoindoline-2-carbonyl)phenyl)methanone (27): ^{l}H NMR (400 MHz, CDCl₃) δ 11.48 (d, J=6.0 Hz, 1H), 7.97 (d, J=2.2 Hz, 1H), 7.66 (t, J=8.2 Hz, 1H), 7.36-7.19 (m, 1H), 7.16-6.92 (m, 3H), 6.88-6.69 (m, 3H), 5.10-4.85 (m, 8H), 3.79 (d, J=7.2 Hz, 3H). ¹³C NMR (101 MHz, CDCl₃) δ 169.8, 169.5, 162.2, 162.1, 161.9, 139.7, 137.8, 136.7, 132.0, 131.9, 130.3, 130.0, 128.3, 128.3, 126.6, 126.2, 126.0, 123.5, 123.4, 118.7, 118.2, 118.0, 117.9, 117.8, 117.0, 114.3, 114.2, 114.1, 107.9, 107.6, 55.6, 55.4, 54.9, 53.7, 53.6, 53.2, 52.3, 52.3, 51.6, 50.1.

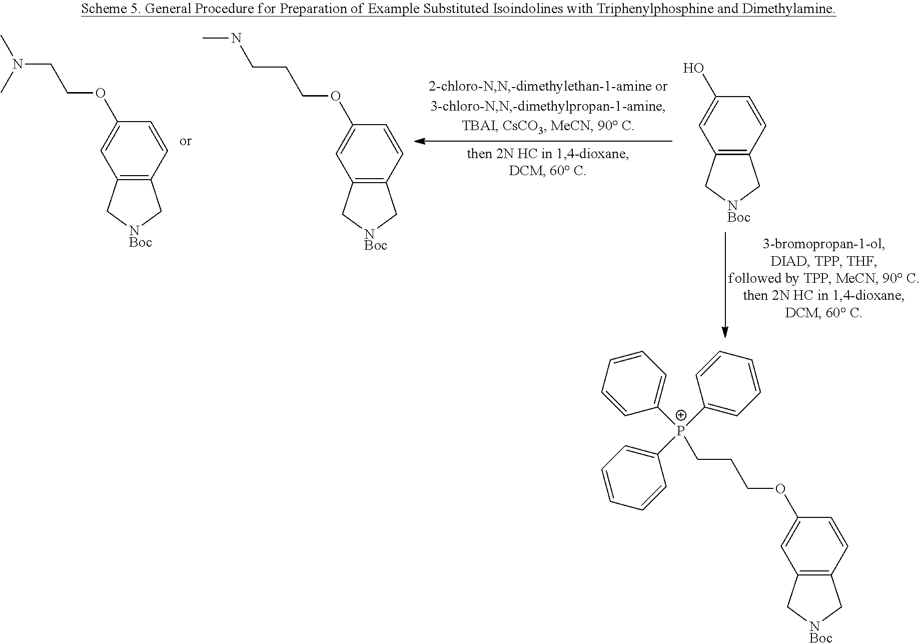 [SMILES: CN(C)CCOc1ccc2c(c1)CN(C(=O)OC(C)(C)C)C2 ; CNCCCOc1ccc2c(c1)CN(C(=O)OC(C)(C)C)C2 ; CC(C)(C)OC(=O)N1Cc2ccc(O)cc2C1 ; CC(C)(C)OC(=O)N1Cc2ccc(OCCC[P+](c3ccccc3)(c3ccccc3)c3ccccc3)cc2C1]

Corresponding benzoic acid (160 μmol), HOBt (479 mol), EDC (479 μmol) and the corresponding isoindoline (239 μmol) were dissolved in dry dichloromethane (0.2 M). DIPEA (958 μmol) was added, and the solution was stirred at rt under argon for 16 h. The solution was taken up in DCM and water, and the aqueous layer was extracted with DCM 2×. For isoindolines containing triphenylphosphine, the combined organic layers were washed with 2 N HCl, and saturated aqueous NaCl solution before being dried with saturated sodium sulfate, filtered and eluent concentrated in vacuo. The crude mixture was redissolved in a minimal amount of DCM, and Et₂O was added. The resulting solid was filtered and washed with more Et₂O. The solid was purified by column chromatography (DCM/MeOH 95/5). Pure product was redissolved in a minimal amount of DCM and washed with 0.1 M aqueous NH₄PF₆. Concentration followed by trituration from diethyl ether afforded compounds as hexafluorophosphate salts. For isodinolines containing dimethylamine, the combined organic layers were washed with saturated aqueous sodium bicarbonate, and saturated aqueous NaCl solution before being dried with saturated sodium sulfate, filtered and eluent concentrated in vacuo. The mixture was purified by column chromatography (DCM/MeOH/NH₄OH 94/5/1).

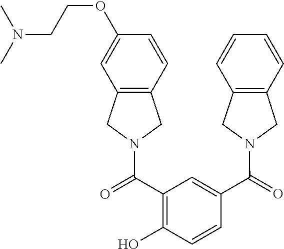 [SMILES: CN(C)CCOc1ccc2c(c1)CN(C(=O)c1cc(C(=O)N3Cc4ccccc4C3)ccc1O)C2]

(5-(2-(Dimethylamino)ethoxy)isoindolin-2-yl)(2-hydroxy-5-(isoindoline-2-carbonyl)phenyl)methanone (28): ¹H NMR (400 MHz, Methanol-d₄) δ 7.71-7.62 (m, 2H), 7.43-7.18 (m, 5H), 7.11-6.92 (m, 3H), 4.96 (d, J=11.8 Hz, 7H), 4.78 (d, J=17.9 Hz, 2H), 4.36 (dt, J=14.3, 5.0 Hz, 2H), 3.61 (dt, J=9.7, 4.9 Hz, 2H), 3.00 (d, J=7.4 Hz, 6H). ¹³C NMR (101 MHz, MeOD) δ 170.24, 155.63, 136.46, 135.63, 130.31, 127.50, 127.33, 123.77, 122.44, 122.26, 115.76, 108.51, 108.29, 61.92, 56.32, 54.78, 52.60, 52.35, 51.32, 48.25, 48.10, 48.03, 47.89, 47.82, 47.68, 47.61, 47.39, 47.18, 46.97, 42.45.

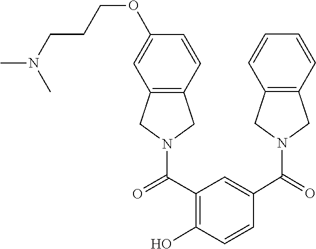 [SMILES: CN(C)CCCOc1ccc2c(c1)CN(C(=O)c1cc(C(=O)N3Cc4ccccc4C3)ccc1O)C2]

(5-(3-(Dimethylamino)propoxy)isoindolin-2-yl)(2-hydroxy-5-(isoindoline-2-carbonyl)phenyl)methanone (29): ¹H NMR (400 MHz, CDCl₃) δ 11.48 (s, 1H), 7.97 (d, J=2.1 Hz, 1H), 7.66 (dd, J=8.5, 2.1 Hz, 1H), 7.39-7.27 (m, 4H), 7.20 (d, J=7.2 Hz, 1H), 7.08 (d, J=8.6 Hz, 1H), 6.87-6.71 (m, 2H), 5.05 (s, 6H), 4.87 (s, 2H), 4.00 (d, J=8.7 Hz, 2H), 2.47 (t, J=7.3 Hz, 2H), 2.27 (s, 6H), 1.97 (q, J=6.8 Hz, 2H).

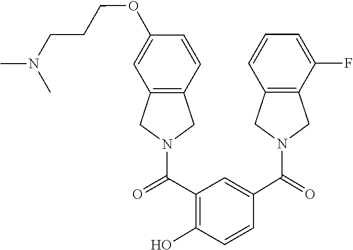 [SMILES: CN(C)CCCOc1ccc2c(c1)CN(C(=O)c1cc(C(=O)N3Cc4cccc(F)c4C3)ccc1O)C2]

(5-(3-(Dimethylamino)propoxy)isoindolin-2-yl)(5-(4-fluoroisoindoline-2-carbonyl)-2-hydroxyphenyl)methanone(30): ¹H NMR (400 MHz, Chloroform-d) δ 7.96 (d, J=2.2 Hz, 1H), 7.66 (t, J=7.6 Hz, 1H), 7.31 (dd, J=14.7, 7.9 Hz, 1H), 7.24-6.93 (m, 4H), 6.80 (d, J=37.0 Hz, 2H), 5.06 (t, J=6.9 Hz, 6H), 4.90 (d, J=12.9 Hz, 2H), 4.00 (s, 2H), 2.51 (t, J=7.3 Hz, 2H), 2.30 (s, 6H), 1.98 (p, J=6.7 Hz, 2H). ¹³C NMR (101 MHz, CDCl₃) δ 169.78, 169.48 (rotamers), 162.09, 161.97 (rotamers), 159.39, 139.76, 132.00, 131.84 (rotamers), 130.26, 130.03 (rotamers), 128.29, 127.99 (rotamers), 126.29, 126.08, 118.74, 118.25, 118.02, 117.91 (rotamers), 114.65, 114.46, 114.29, 114.09, 108.28, 77.48, 77.16, 76.84, 66.53, 56.40, 55.44, 53.72, 53.16, 52.32, 50.06, 45.48, 27.39.

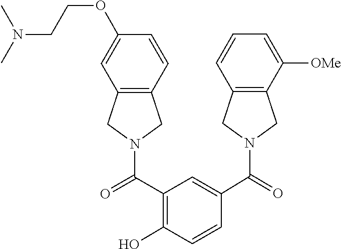 [SMILES: COc1cccc2c1CN(C(=O)c1ccc(O)c(C(=O)N3Cc4ccc(OCCN(C)C)cc4C3)c1)C2]

(5-(2-(Dimethylamino)ethoxy)isoindolin-2-yl)(2-hydroxy-5-(4-methoxyisoindoline-2-carbonyl)phenyl)methanone(31): ¹H NMR (400 MHz, CDCl₃) δ 11.41 (s, 1H), 7.96 (d, J=2.2 Hz, 1H), 7.66 (ddd, J=10.9, 8.3, 2.1 Hz, 1H), 7.33-7.03 (m, 3H), 6.97-6.73 (m, 4H), 5.14-4.94 (m, 6H), 4.82 (d, J=21.7 Hz, 2H), 4.05 (s, 2H), 3.84 (d, J=24.2 Hz, 3H), 2.73 (t, J=5.5 Hz, 2H), 2.34 (s, 6H). ¹³C NMR (101 MHz, CDCl₃) δ 169.9, 169.5 and 169.4 (rotamers), 162.0, 155.2, 154.7, 138.2 and 138.1 (rotamers), 132.2 and 131.9 (rotamers), 129.8, 129.5, 128.1, 126.7, 126.5, 124.7, 123.5, 118.0 and 117.9 (rotamers), 116.8, 115.0, 114.5, 109.2, 108.8, 66.4, 58.4, 55.8, 55.5 and 55.3 (rotamers), 53.7, 53.4 and 53.4 (rotamers), 51.0, 46.0.

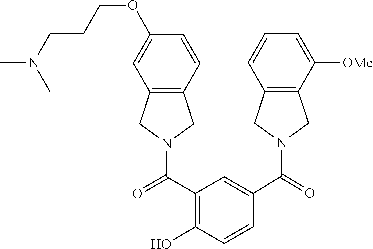 [SMILES: COc1cccc2c1CN(C(=O)c1ccc(O)c(C(=O)N3Cc4ccc(OCCCN(C)C)cc4C3)c1)C2]

(5-(3-(Dimethylamino)propoxy)isoindolin-2-yl)(2-hydroxy-5-(4-methoxyisoindoline-2-carbonyl)phenyl)methanone(32): ¹H NMR (400 MHz, CDCl₃) δ 7.97 (d, J=2.1 Hz, 1H), 7.67 (dt, J=8.2, 1.7 Hz, 1H), 7.34-7.28 (m, 2H), 7.08 (dd, J=8.6, 4.0 Hz, 1H), 6.97-6.73 (m, 4H), 5.16-4.93 (m, 6H), 4.83 (d, J=21.1 Hz, 2H), 4.01 (s, 2H), 3.84 (d, J=24.3 Hz, 3H), 2.49 (s, 2H), 2.29 (s, 6H), 1.98 (t, J=7.9 Hz, 2H). ¹³C NMR (101 MHz, CDCl₃) δ 169.9 and 169.5 (rotamers), 162.0 and 161.9 (rotamers), 154.7, 138.2, 138.1, 132.2, 131.9, 129.8, 129.5, 128.1, 126.7, 126.5, 124.7, 118.0, 117.9, 116.8, 115.0, 114.5, 109.2, 108.8, 66.6, 56.4, 55.8, 55.5, 55.3, 53.4 and 53.4 (rotamers), 51.0, 45.6, 27.5.

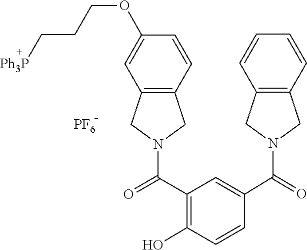

(3-((2-(2-Hydroxy-5-(isoindoline-2-carbonyl)benzoyl)isoindolin-5-yl)oxy)propyl)triphenylphosphonium hexafluorophosphate(V)(33): ¹H NMR (400 MHz, CDCl₃) δ 11.41 (s, 1H), 7.92 (d, J=2.0 Hz, 1H), 7.85-7.60 (m, 17H), 7.40-7.29 (m, 2H), 7.24-7.00 (m, 3H), 6.89-6.61 (m, 2H), 5.11-4.83 (m, 8H), 4.14 (s, 2H), 3.41 (t, J=7.2 Hz, 2H), 2.14 (s, 2H). ¹³C NMR (101 MHz, CDCl₃) δ 169.7, 169.6, 161.6, 136.5, 136.2, 135.5, 135.5, 133.6, 133.5, 131.9, 130.9, 130.7, 128.2, 128.0, 127.8, 126.5, 123.5, 123.0, 122.7, 118.3, 117.8, 117.4, 114.9, 108.6, 66.8, 66.6, 55.4, 52.9, 22.8, 19.6, 19.1. ¹P NMR (162 MHz, CDCl₃) δ 24.13, −135.52, −139.92, −144.33, −148.73, −153.13.

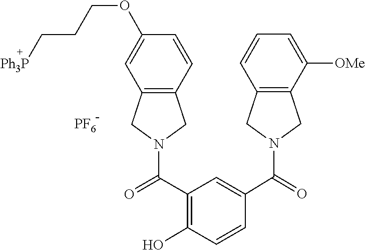

(3-((2-(2-Hydroxy-5-(4-methoxyisoindoline-2-carbonyl)benzoyl)isoindolin-5-yl)oxy)propyl)triphenylphosphonium hexafluorophosphate(V)(34): ¹H NMR (400 MHz, Chloroform-d) δ 11.41 (s, 1H), 7.90 (t, J=2.6 Hz, 1H), 7.79 (tp, J=7.0, 4.2, 3.3 Hz, 3H), 7.75-7.59 (m, 13H), 7.29 (d, J=8.0 Hz, 1H), 7.20-6.97 (m, 2H), 6.95-6.61 (m, 4H), 4.96 (t, J=18.1 Hz, 6H), 4.81 (d, J=22.1 Hz, 2H), 4.14 (s, 2H), 3.82 (d, J=21.9 Hz, 3H), 3.41 (ddd, J=16.3, 10.5, 6.4 Hz, 2H), 2.13 (q, J=8.2, 7.5 Hz, 2H). ¹³C NMR (101 MHz, CDCl₃) δ 169.70, 169.64 (rotamers), 169.48, 169.39 (rotamers), 161.58, 161.41 (rotamers), 155.14, 154.71 (rotamers), 138.19, 137.98 (rotamers), 135.49, 135.46 (rotamers), 133.54, 133.44 (rotamers), 132.07, 131.82, 130.84, 130.71 (rotamers), 129.78, 129.53, 128.09, 126.74, 126.57 (rotamers), 124.62, 118.23, 117.88, 117.75, 117.37, 117.16, 114.92, 114.53, 109.18, 108.83 (rotamers), 108.60, 66.77, 66.61 (rotamers), 55.71, 55.49, 55.36 (rotamers), 53.57, 53.40, 53.35 (rotamers), 51.05, 22.82, 22.79 (rotamers), 19.62, 19.08 (rotamers). ³¹P NMR (162 MHz, CDCl₃) δ 24.09, −131.13, −135.53, −139.93, −144.34, −148.74, −153.14, −157.54.

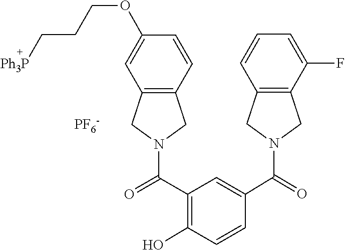

(3-((2-(5-(4-Fluoroisoindoline-2-carbonyl)-2-hydroxybenzoyl)isoindolin-5-yl)oxy)propyl)triphenylphosphonium hexafluorophosphate(V)(35): ¹H NMR (400 MHz, Chloroform-d) δ 11.44 (d, J=16.3 Hz, 1H), 7.91 (s, 1H), 7.79 (tp, J=6.8, 4.0, 3.2 Hz, 3H), 7.74-7.60 (m, 13H), 7.29 (d, J=7.6 Hz, 1H), 7.18-6.87 (m, 4H), 6.87-6.67 (m, 2H), 5.04 (s, 2H), 4.91 (t, J=10.5 Hz, 6H), 4.13 (s, 2H), 3.39 (td, J=13.5, 13.1, 7.2 Hz, 2H), 2.18-2.05 (m, 2H). ³¹P NMR (162 MHz, CDCl₃) δ 24.07, −131.13, −135.53, −139.93, −144.34, −148.74, −153.14, −157.54.

### 2. Biological Evaluation of Compounds

Fluorescence polarization assays. The FP assay was performed in 96-well format in black, flat-bottom plates (Santa Cruz Biotechnology) with a final volume of 100 μL. Twenty-five microliters of assay buffer (20 mM HEPES, 50 mM KCl, 10.5 mM MgCl₂, 20 mM Na₂MoO₄, 0.010% NP-40 detergent (NP-40), and pH 7.3 with fresh 2 mM dithiothreitol (DTT) and 0.1 mg mL⁻¹ bovine γ-globulin (BGG) added before use), 25 μL of assay buffer containing 6 nM FITC-GDA (fluorescent tracer, stock in DMSO and diluted in assay buffer) and 50 μL of assay buffer containing 10 nM of Hsp90, Hsp90a, Grp94, or Trap1 were added to each well. Compounds were tested in triplicate wells (1% DMSO final concentration). For each plate, wells containing buffer only (background), tracer in buffer only (low polarization control), and protein and tracer in buffer with 1% DMSO (high polarization control) were included. Plates were incubated at 4° C. with rocking for 24 h. Polarization values (in mP units) were measured at 37° C. with an excitation filter at 485 nm and an emission filter at 528 nm. Polarization values were correlated to % tracer bound and compound concentrations. The concentration at which the tracer was 50% displaced by the compound of interest were calculated and reported as K_{d} values.

Anti-proliferation assays. Cells (2,000/well; 150 μL volume) were seeded in black, cell culture-treated 96-well plates with clear bottom and incubated overnight at 37° C. Varying concentrations of compounds dissolved in DMSO (1% final concentration) were added to cells, and the treated cells were returned to incubate for 72 hr while a separate cohort of cells were subject to a Day Zero read using an MTS/phenazine methosulfate (PMS) antiproliferation kit (Promega) according to the manufacturer's instructions. An Epoch 96-well plate reader to measure absorbance at 490 nm. After 72 hr, the treated cells were subject to the same protocol using the same reagents. Absorption values from 1% DMSO—Day Zero were used as 100% proliferation, and the resulting dose-response data were used to determine anti-proliferative IC₅₀ values using GraphPad Prism software.

Western blots for protein degradation. Cells were incubated with drugs or vehicle (DMSO) dissolved in respective media for 6 h. After 6 h of drug exposure, the cells were washed with 1×PBS (137 mM NaCl, 2.7 mM KCl, 10 mM Na₂PO₄, 1.8 mM KH₂PH₄, pH 7.4) and then lysed in RIPA buffer (10 mM Tris-Cl, pH 8.0, 130 mM NaCl, 1 mM EDTA, 1% Triton X-100, 0.10% SDS) containing 1:100 Phosphatase Inhibitor Cocktail 2, 1:100 Phosphatase Inhibitor Cocktail 3 (Sigma-Aldrich, St. Louis, MO, USA), 1:100 Protease Inhibitor Cocktail Set III, EDTA-Free (Calbiochem, MilliporeSigma, Burlington, MA, USA), and 1 mM phenylmethyl sulfonyl fluoride. The collected cell lysates were centrifuged at 10,000 rcf for 10 min at 4° C. Supernatants were collected and subject to BCA assay (Pierce™ BCA Protein Assay Kit, Pierce, Thermo Fisher Scientific, Waltham, MA, USA) to determine protein concentration. Samples (30 μg/each) were loaded into the wells of a 4-20% Mini-PROTEAN TGX™ Precast Gel (Bio-Rad, Hercules, CA, USA), electrophoresed at 180 V, and transferred to either an Immun-Blot PVDF Membrane or a nitrocellulose membrane using a Trans-Blot Turbom Transfer System (Bio-Rad, Hercules, CA, USA). Membranes were blocked in 7% non-fat milk for 1 h at rt prior to exposure to primary antibody solutions. Primary antibodies used in these experiments included Anti-NDUFS1 Antibody (E-8, Santa Cruz; 1:500), Glutaminase-1/GLS-1 (88964, Cell Signaling; 1:500), Anti-SIRT3 Antibody (F-10, Santa Cruz; 1:500), Akt (9272, Cell Signaling; 1:1000), TRAP1 (9B6, Enzo Life Sciences; 1:1000), CDK4 (D9G3E, 12790, Cell Signaling; 1:1000), HSP70/HSP72 (C92F3A-5, Enzo Life Sciences, 1:1000), and β-actin (8H10D10, 3700, Cell Signaling, 1:1000). Each antibody solution consisted of 0.3% sodium azide in 7% non-fat milk. Secondary antibodies used in these experiments were Anti-rabbit IgG, HRP-linked antibody (1:2000, Cell Signaling) and Goat Anti-mouse Ig, Human ads-HRP (1:2000, SouthemBiotech). Blots were visualized using a ChemiDoc Imaging System (Bio-Rad, Hercules, CA, USA).

Seahorse XFe analysis for mitochondrial function. XFe96 Sensor Cartridge was placed in a humidified 37° C. incubator without CO₂ overnight with 200 μL of molecular biology grade water in each well of the XF Utility Plate. Cells were then seeded (HeLa: 30,000 cells/well; 22Rv1: 50,000 cells/well) into a XF96 Cell Culture Microplate (Agilent Technologies) and allowed to adhere in their respective culture growth media (described above) overnight. Assay media—Seahorse DMEM (HeLa) or Seahorse XF RPMI 1640 (22Rv1) supplemented with 1 mM pyruvate, 2 mM glutamine, and 10 mM glucose—was prepared fresh for each experiment. Cells were washed with assay media and then incubated with drug or vehicle (DMSO) dissolved in assay media for 6 h in a humidified 37° C. incubator without CO₂. The XFe96 Sensor Cartridge was hydrated with 200 μL/well of XF Calibrant for 2 h prior to each experiment. Data analyses were performed using GraphPad Prism software.

Molecular cloning, protein expression, and purification. Human TRAP1_70-552 was cloned into a modified pET expression plasmid with an N-terminal 6×His-tag, including a Tobacco Etch Virus (TEV) protease cleavage site between the tag and the protein sequence. Expression of the protein was carried out in E. coli BL21 and induced with 0.2 mM IPTG at 18° C. overnight. The recombinant protein was purified by a two-step Ni-NTA affinity chromatography. The elution protein from the first Ni-NTA affinity column step was subsequently subjected to TEV protease cleavage at 1:100 mass ratio, which was dialyzed against loading buffer (20 mM Tris-HCl, 500 mM sodium chloride, 20 mM imidazole, pH 8.0) at 4° C. overnight. The protein was then collected as flow through from a second subtracting Ni-NTA column. The protein was further purified by size exclusion chromatography to homogeneity with a buffer containing 20 mM Tris PH8.0, 150 mM NaCl, 1 mM TCEP. The protein was finally concentrated to 20 mg/mL for crystallization.

Crystallization and data collection. 20 mg/mL hTRAP1_70-552 (hTRAP1_NM) protein was mixed with 1 mM compound 1, compound 7 and compound 26 respectively, which were incubated on ice for 1 hour before crystallization. Crystallization screening was performed and crystal of hTRAP1_NM-compound 1 complex was obtained in the condition of 0.1 M Sodium malonate PH6.0, 12% PEG3350 at 4° C. Crystal of hTRAP1_NM-compound 7 complex appeared in the condition of 0.1 M Sodium malonate PH6.0, 12% PEG3350 at room temperature. hTRAP1_NM-compound 26 complex was crystallized in the condition of 1% w/v tryptone, 0.05M Hepes-Na PH7.0, 12% PEG3350 at room temperature. 20% glycerol was added to the mother liquid as cryoprotectant before flash frozen in liquid nitrogen. All data were collected from the beamline 19-ID at the Advanced Photon Source, Argonne National Laboratory.

Structural Determination. Diffraction data were processed using HKL-3000. The initial structure model was solved by the molecular replacement method using phaser, with PDB SHPH as the template. The PHENIX software program was used for structural refinement. Electronic Ligand Builder and Optimization Workbench (eLBOW) was used to generate the ligand restraints for refinement. Translation, libration and screw-rotation (TLS) displacement groups used in the refinement were defined by the TLMSD server. COOT was used for the iterative manual structural building. The final R_{work} and R_{free} for the refined models of hTRAP1_NM with three compounds are listed respectively in Table4 The current models are of good geometries and refinement statistics (Table 4). All molecular graphic figures were generated with PYMOL [8]. The structures were deposited into the protein data bank with accession codes 7U8V (compound 5), 7U8W (compound 20), and 7U8X (compound 27).

### Example 1: Evaluation of Affinities for TRAP1-Selective Inhibitors

Radicicol-based isoform-selective inhibitors were obtained through phenol removal on the resorcinol moiety and solution of their resulting co-crystal structures with Hsp90α and Hsp90β (Mishra et al., 2021; Khandelwal et al., 2018). Unfortunately, only a few TRAP1 co-crystal structures exist, and those are bound to purine-based small molecules, highlighting the lack of structural information for the development of new TRAP1 inhibitors. Thus, efforts to develop TRAP1 N-terminal inhibitors commenced by screening a small library (˜170) of radicicol-based Hsp90 inhibitors. Compounds with an isoindoline, including hit compound 1, demonstrated sub-micromolar TRAP1 affinity and subsequent modification of the resorcinol moiety led to phenols 2 and 3, which provided some evidence for TRAP1 selectivity versus the other isoforms. Molecular modeling studies suggested the indole was solvent exposed and subsequent modifications did not lead to inhibitors with improved affinity. Therefore, compound 4 was pursued as a symmetrical bis-isoindoline analog of 1, whereas compound 5 was developed from the pan-inhibitor 4 via the removal of a phenol, which exhibited a modest 13-fold selectivity for TRAP1 versus Grp94 and did not display measurable affinity for either cytosolic isoform (Table 1). A detailed structural analysis of compound 5 bound to the ATP-binding pocket of TRAP1_NM is given in Table 4.

TABLE 1
Development of TRAP1-selective inhibitor scaffolds

| | 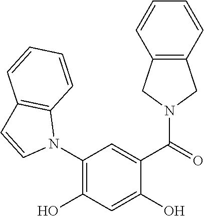 [SMILES: O=C(c1cc(-n2ccc3ccccc32)c(O)cc1O)N1Cc2ccccc2C1] | 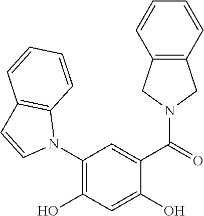 [SMILES: O=C(c1cc(-n2ccc3ccccc32)c(O)cc1O)N1Cc2ccccc2C1] | 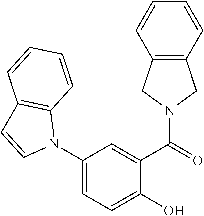 [SMILES: O=C(c1cc(-n2ccc3ccccc32)ccc1O)N1Cc2ccccc2C1] |
|---|---|---|---|
| | 1 | 2 | 3 |
| TRAP1 (μM) | 0.42 ± 0.1 | 2.5 ± 0.05 | >100 |
| GPR94 (μM) | 0.32 ± 0.02 | >100 | >100 |
| Hsp90α (μM) | 0.2 ± 0.05 | >100 | n.d. |
| Hsp90β (μM) | 0.47 ± 0.02 | >100 | n.d. |
| | | 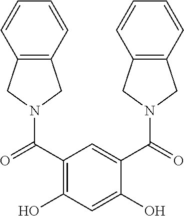 [SMILES: O=C(c1cc(C(=O)N2Cc3ccccc3C2)c(O)cc1O)N1Cc2ccccc2C1] | 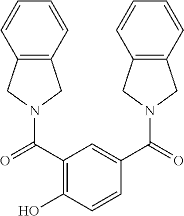 [SMILES: O=C(c1ccc(O)c(C(=O)N2Cc3ccccc3C2)c1)N1Cc2ccccc2C1] |
| | | 4 | 5 |
| | TRAP1 (μM) | 0.44 ± 0.02 | 2.62 ± 0.47 |
| | GPR94 (μM) | 0.46 ± 0.07 | 34.72 ± 5.8 |
| | Hsp90α (μM) | 0.45 ± 0.03 | >100 |
| | Hsp90β (μM) | 1.00 ± 0.05 | >100 |

The studies focused on the identification of optimal substituents about the two isoindolines for potency against TRAP1, as well as to further probe SAR trends for selectively against Grp94 and Hsp90a. For example, intermediates 7-14 were synthesized via an amide coupling reaction between 4-hydroxy-3-(methoxycarbonyl)benzoic acid (6) with the respective isoindoline, followed by hydrolysis of the methyl ester. A second amide coupling reaction was then preformed with the intermediate carboxylic acid and substituted isoindoline to yield compounds 15-36.

Compounds 15-21 contained mono- or bis-fluro-isoindoline derivatives of 5, which manifested 2.62 μM binding affinity for TRAP1. These seven compounds provided evidence to support further derivatization at R¹, R² and R⁴ but not R³. As shown in Table 1, analogs that contain a fluorine at R³ (15 and 19) manifested >2.62 μM TRAP1 affinity. Molecular modeling studies suggested that R³ sterically clashes with Trp231 within the binding pocket and disrupts π-stacking interactions, suggesting that R³ cannot tolerate derivatization without compromising TRAP1 binding.

Compounds 16, 17, 20 and 21 contain fluorine substitution(s) at R¹ and/or R⁴ and all displayed improved TRAP1 binding affinity as compared to 5. Furthermore, 16, 20, and 21 displayed a trend suggesting that R⁴ could be modified for additional TRAP1 affinity, while R¹ or R² were solvent exposed. Therefore, R⁴ was diversified to contain a chlorine, bromine, methyl, methoxy or ethoxy moiety, which ultimately led to the identification of 25 as a 40 nM TRAP1 inhibitor.

Compound 27 was synthesized, wherein the R² fluorine was replaced with a methoxy group. It was hypothesized that the oxygen would interact favorably with Gln200 outside the binding pocket and serve as a handle for which moieties to increase cell permeability could be attached for mitochondrial localization. Since 27 retained good selectivity and reasonable affinity, analogs of 27 were synthesized wherein the methyl ether was replaced with a N,N-dimethylaminoethyl (A), N,N-dimethylaminopropyl (B) or a triphenylphosphonium (C) group (Table 2) in an effort to enhance the mitochondrial accumulation. This strategy resulted in compounds that improved anti-proliferative activities against several cell lines (Table 3).

TABLE 2
SAR of isoindoline TRAP1 inhibitors

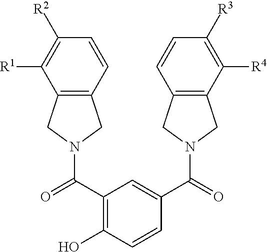

| Entry | R¹ | R² | R³ | R⁴ | Hsp90α (μM) | Grp94 (μM) | TRAP1 (μM) |
|---|---|---|---|---|---|---|---|
| PU-H71 | — | — | — | — | — | 0.03 ± 0.01 | 0.24 ± 0.04 |
| 4 | — | — | — | — | 0.45 ± 0.03 | 0.4 ± 0.07 | 0.43 ± 0.02 |
| 5 | H | H | H | H | >100 | 34.72 ± 5.80 | 2.63 ± 0.47 |
| 15 | H | H | F | H | >100 | 48.48 ± 2.15 | 24.04 ± 0.14 |
| 16 | H | H | H | F | >100 | 35.03 ± 2.07 | 0.89 ± 0.06 |
| 17 | F | H | H | H | >100 | 30.34 ± 3.83 | 0.96 ± 0.01 |
| 18 | H | F | H | H | >100 | 30.72 ± 6.76 | 2.43 ± 0.43 |
| 19 | H | F | F | H | >100 | >10 | >5 |
| 20 | H | F | H | F | >100 | >10 | 0.35 ± 0.03 |
| 21 | F | H | H | F | >100 | >10 | 0.36 ± 0.02 |
| 22 | H | H | H | Cl | >100 | >10 | 0.23 ± 0.03 |
| 23 | H | H | H | Br | >100 | >10 | 0.38 ± 0.06 |
| 24 | H | H | H | Me | >100 | >10 | 0.55 ± 0.07 |
| 25 | H | H | H | OCH₃ | >100 | 3.90 ± 0.42 | 0.04 ± 0.01 |
| 26 | H | H | H | OCH₂CH₃ | >100 | 6.50 ± 1.70 | 0.08 ± 0.01 |
| 27 | H | OCH₃ | H | F | >100 | >10 | 0.47 ± 0.02 |
| 28 | H | | H | H | — | >10 | 0.93 ± 0.01 |
| 29 | H | | H | F | — | >10 | 0.49 ± 0.03 |
| 30 | H | | H | OMe | — | 4.76 ± 0.43 | 0.09 ± 0.02 |
| 31 | H | | H | H | — | 5.50 ± 0.96 | 0.86 ± 0.03 |
| 32 | H | | H | F | — | 6.94 ± 1.25 | 0.47 ± 0.06 |
| 33 | H | | H | OMe | — | 1.84 ± 0.20 | 0.09 ± 0.01 |
| 34 | H | | H | H | — | >10 | 0.65 ± 0.12 |
| 35 | H | | H | F | — | >10 | 0.16 ± 0.02 |
| 36 | H | | H | OMe | — | >10 | 0.04 ± 0.01 |

### Example 2: Anti-Proliferative Effects of TRAP1 Inhibitors

While some compounds in Table 1 exhibit improved affinity for TRAP1 in vitro when compared to 4 and 5, early studies indicated the compounds did not manifest antiproliferative activity against SK-OV-3 or PC3 cells, which was anticipated for compounds that inhibit TRAP1 in these cell lines (Table 2). These early findings suggest the compounds may be unable to localize into the mitochondria, and this was further supported by the biological evaluation of these molecules reported herein.

TABLE 3
Anti-proliferatives activity (IC₅₀ values) of newly-developed TRAP1
inhibitors as determined by MTS assay against various cell lines.

| | 22RV1 | HeLa | SK-OV-3 | PC3 |
|---|---|---|---|---|
| # | (μM) | (μM) | (μM) | (μM) |
| 15-21 | — | — | >64 | >64 |
| 32 | 8.50 | 23.7 | >64 | 0.440 |
| 35 | 4.27 | 3.83 | — | 2.58 |
| 36 | 10.1 | 16.4 | — | 23.9 |
| MitoQ | 0.381 | 2.48 | — | 1.28 |

### Example 3: Western Blot Analysis of Cellular Activity for TRAP1 Inhibitors

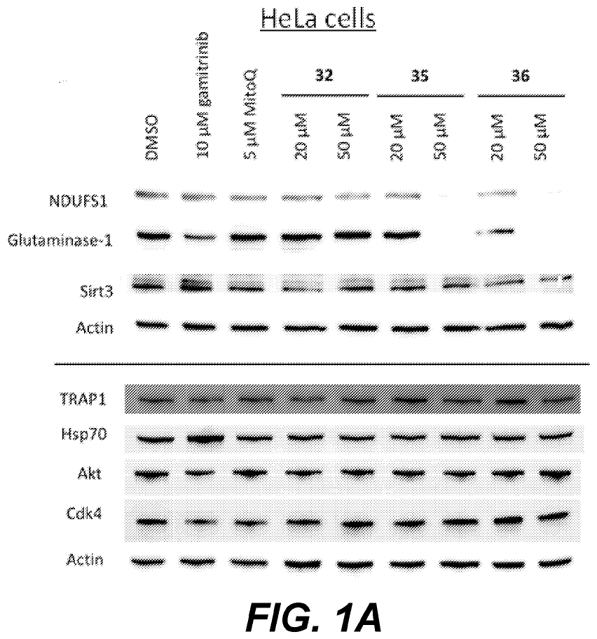

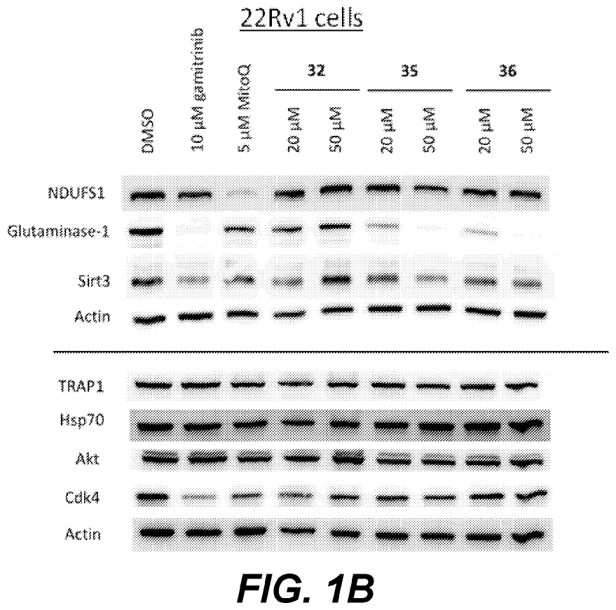

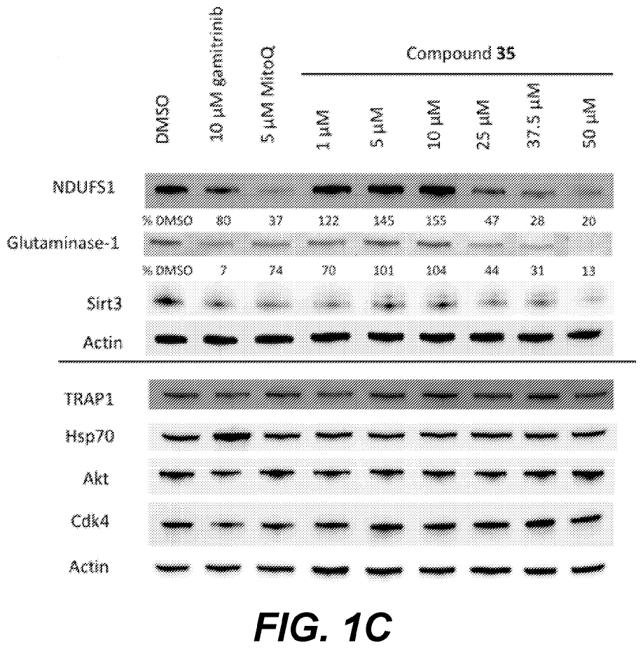

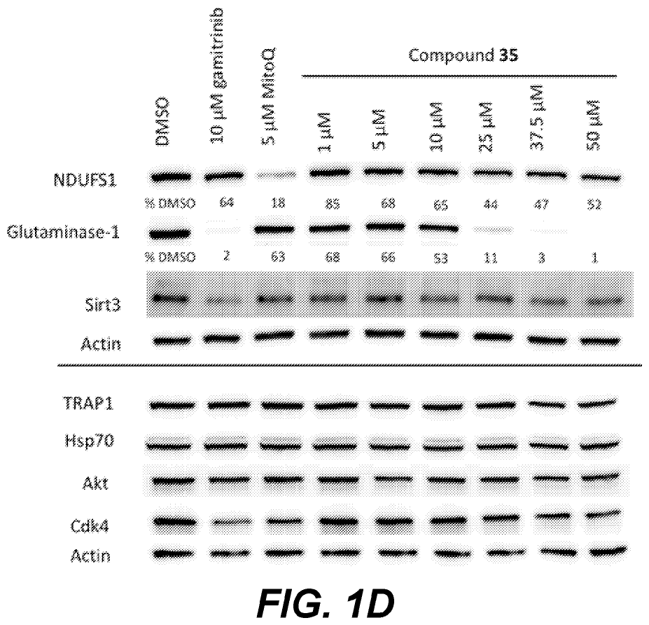

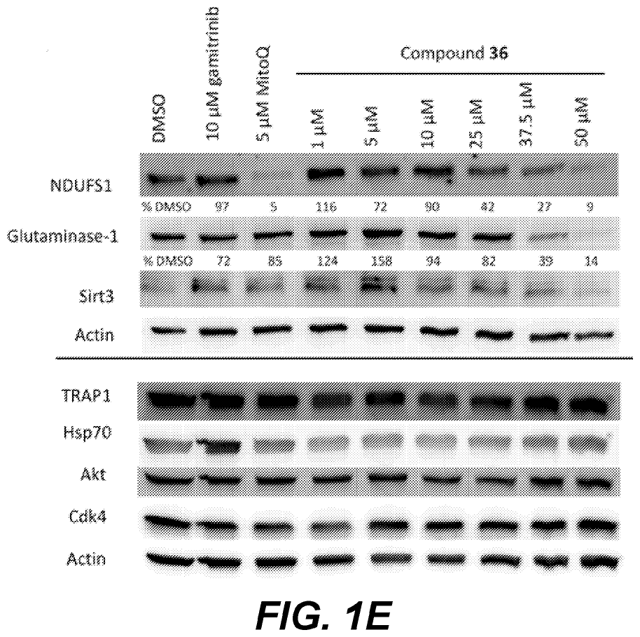

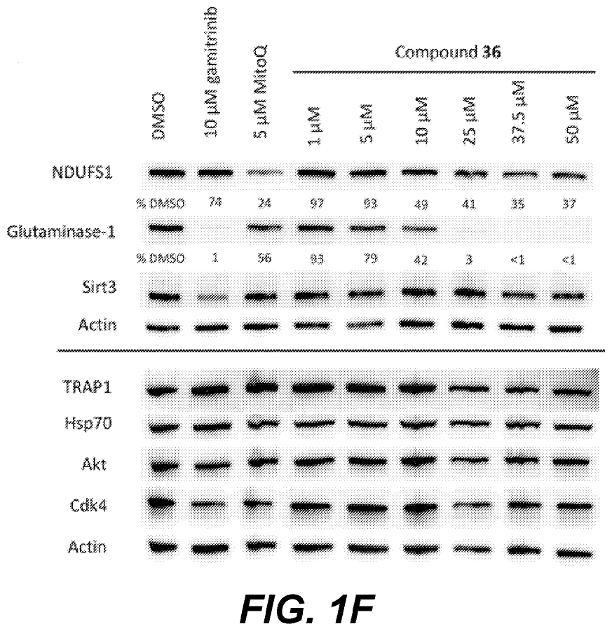

Western blot experiments were performed to demonstrate selective TRAP1 engagement in cells. As can be seen in FIG. 1A-F, TRAP1 inhibitors with the triphenylphosphonium (TPP) moiety, 35 and 36, exhibited dose-dependent and selective degradation of TRAP1-dependent client proteins NDUFS1 (NADH:Ubiquinone Oxidoreductase Core Subunit S1), glutaminase-1, and Sirt3 (Sirtuin 3). This agrees with the activity of MitoQ, a TPP-containing TRAP1 inhibitor that was recently reported by Yoon, N. G. et al. to induce the degradation of these clients. Importantly, the compounds manifested TRAP1 inhibitory activity without inducing the degradation of the cytosolic Hsp90 clients, Akt and Cdk4, or induction of Hsp70, which is an outcome that results from pan-Hsp90 inhibition as exemplified by the TPP-containing Hsp90 inhibitor, gamitrinib. Treatment with the dimethylamine compound 32 did not induce the degradation of TRAP1 client proteins, which suggests the TPP moiety is necessary for mitochondrial localization and/or TRAP1 engagement in these cells.

### Example 4: Effect of TRAP1 Inhibitors on Mitochondrial Function

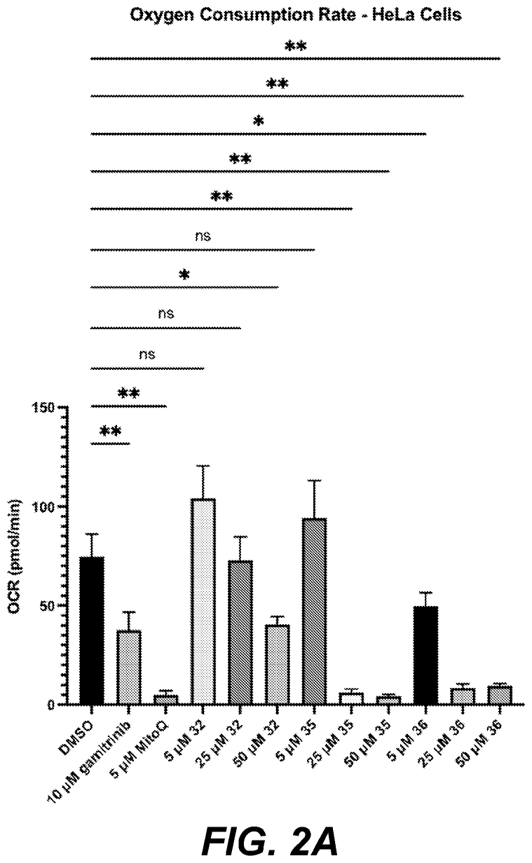

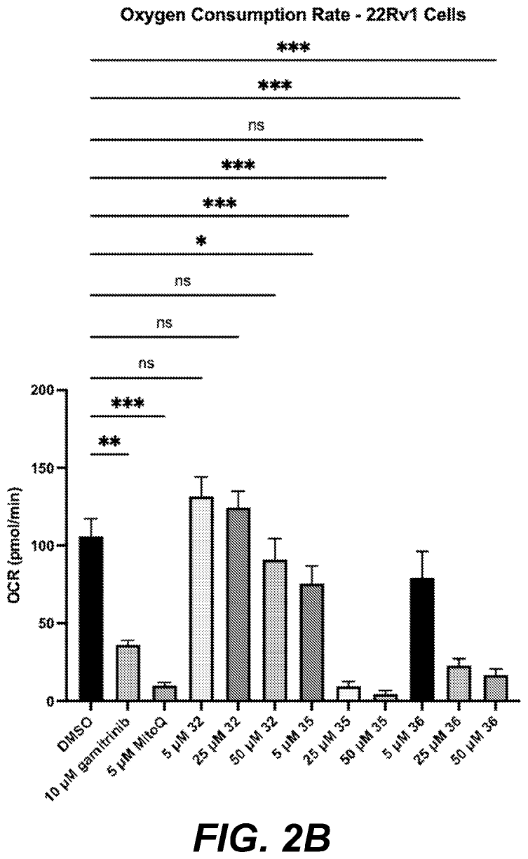

Recent studies have shown that TRAP1 is not only involved in the folding and assembly of mitochondrial client proteins, but also serves to mediate oxidative phosphorylation (OXPHOS), primarily through interactions with complex II and IV of the electron transport chain. Therefore, experiments were performed to measure cellular oxygen consumption rates after 6 hr treatment with the TRAP1 inhibitors. In agreement with a previous report that treatment with gamitrinib decreased OXPHOS in several cell lines, the TPP-containing inhibitors 35 and 36 produced a significant dose-dependent decrease in OXPHOS in both HeLa and 22Rv1 cells (FIG. 2A-2B). The dimethylamine derivative 32, which did not induce client protein degradation in these cells line, did not affect oxidative phosphorylation.

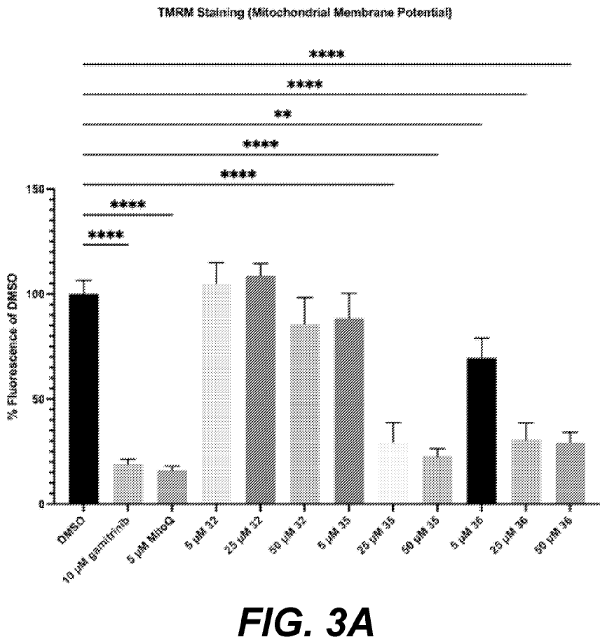

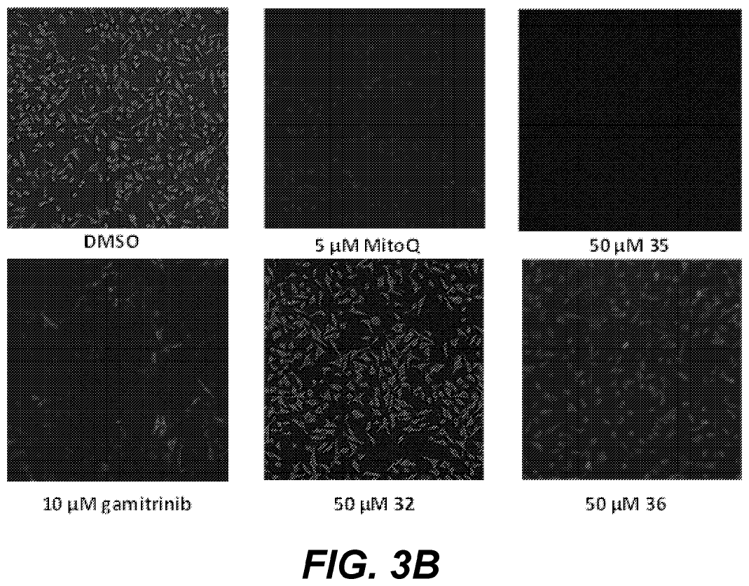

Tetramethylrhodamine (TMRM) is a fluorescent, cell-permeable dye that accumulates in functional mitochondria and is used to measure the mitochondrial membrane potential. Previously, TMRM staining was employed to show a decrease in mitochondrial membrane potential in response to TRAP1 knockdown in primary motor neuron cultures. Similarly, HeLa cells treated with MitoQ, gamitrinib, 35, and 36 exhibited a marked decrease in TMRM fluorescence, suggesting the mitochondrial membrane potential was disrupted as a direct result of TRAP1 inhibition (FIG. 3A-B).

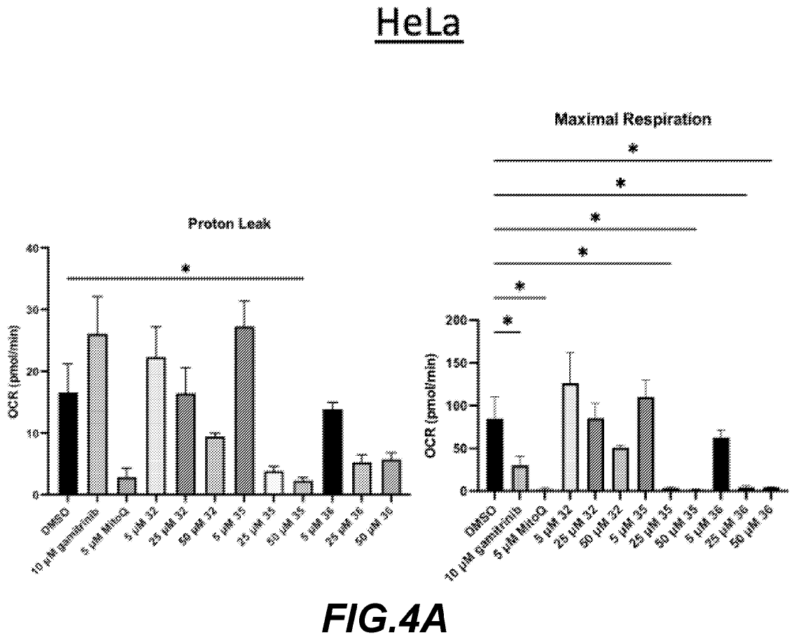

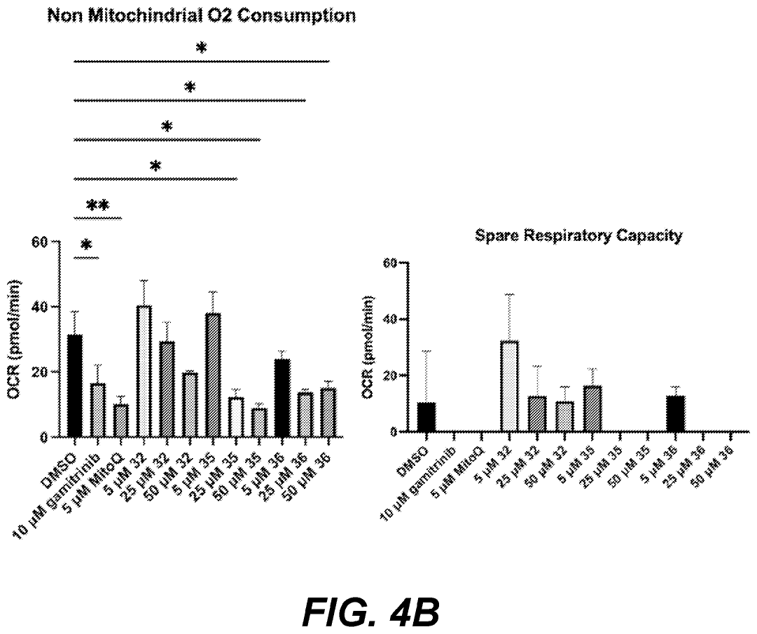

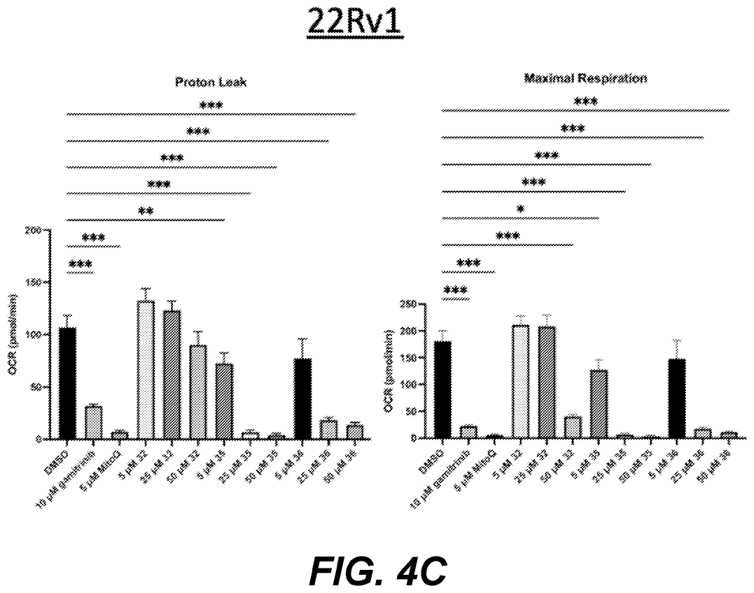

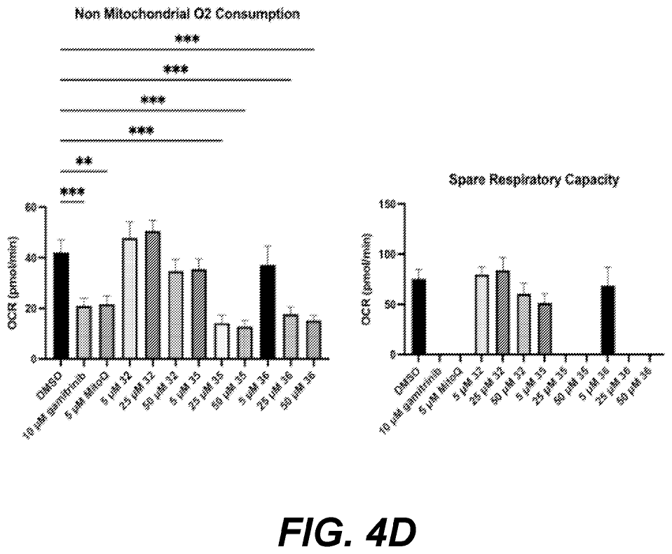

Additional experiments were performed in HeLa and 22Rv1 cells following 6 hr pretreatment with these inhibitors to further assess both mitochondrial and non-mitochondrial function. As displayed in FIG. 4A-D, sequential injections of oligomycin, FCCP, and rotenone/antimycin A (rot/AA) revealed the TPP-containing inhibitors gamitrinib, MitoQ, 35, and 36 decreased proton leak, maximal respiration, non-mitochondrial 02 consumption, and spare respiratory capacity in both cell lines. The dimethylamine compound 32 exhibited minimal activity in these experiments, suggesting that its biological activities may result from off-target engagement. The spare respiratory capacity data were not subjected to ANOVA analyses, as these were non-detectable in response to TPP-containing compounds.

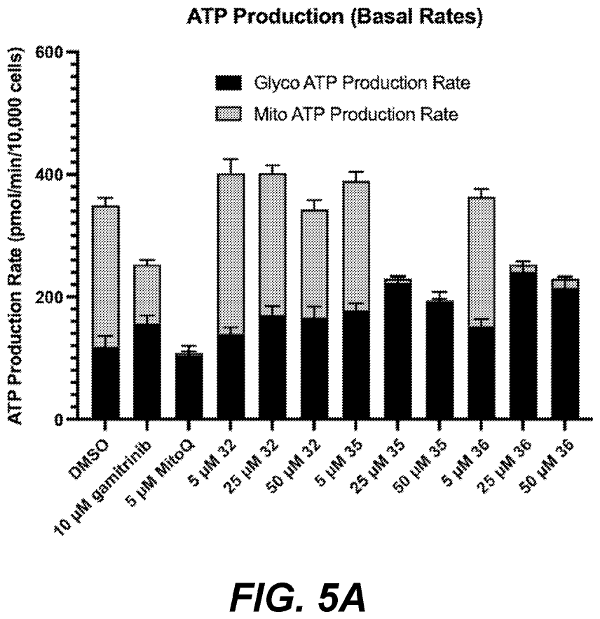

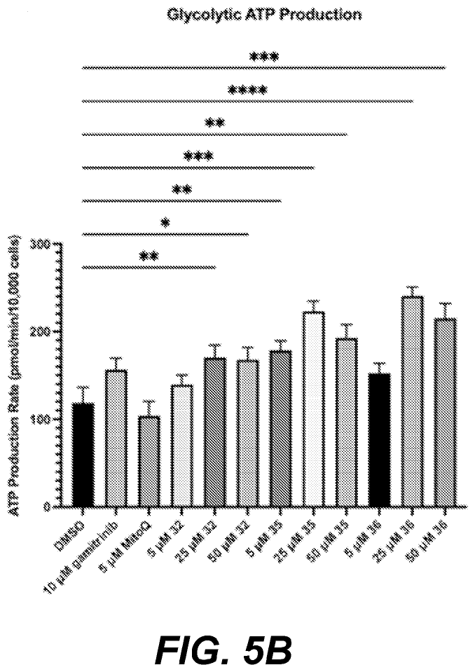

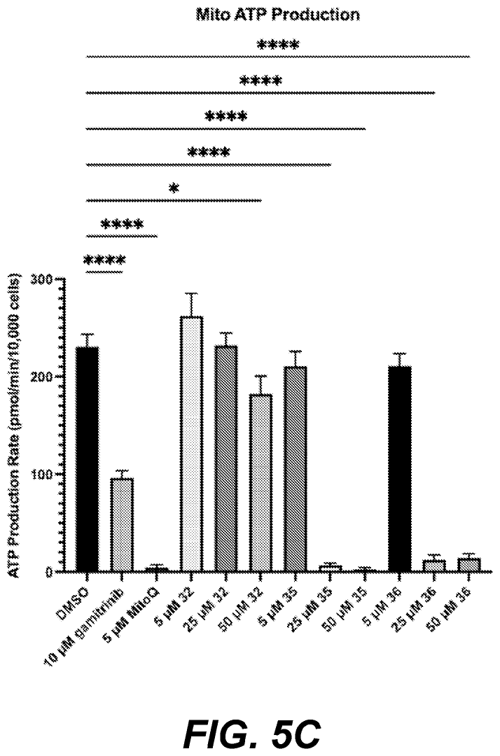

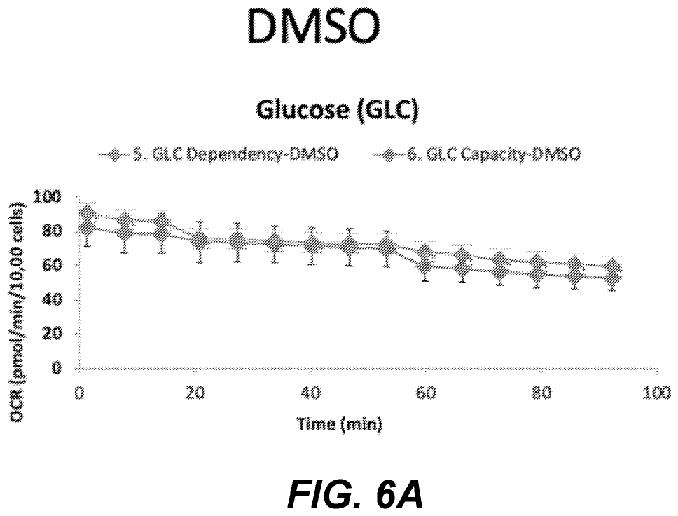

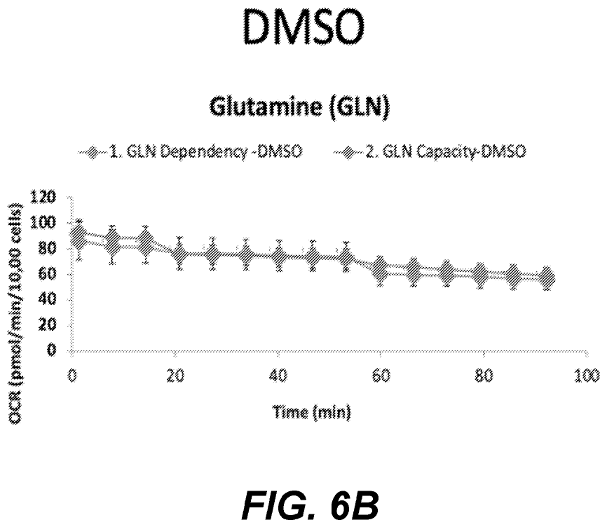

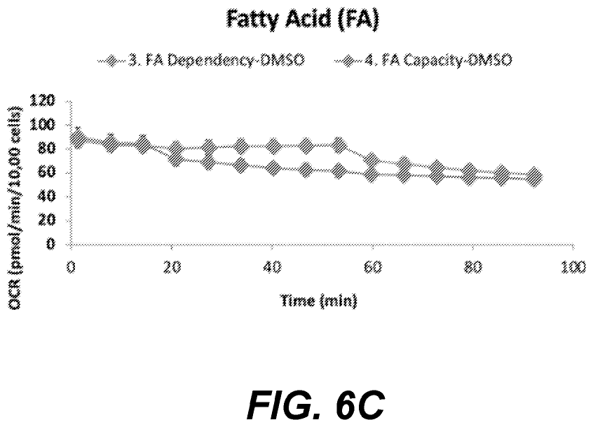

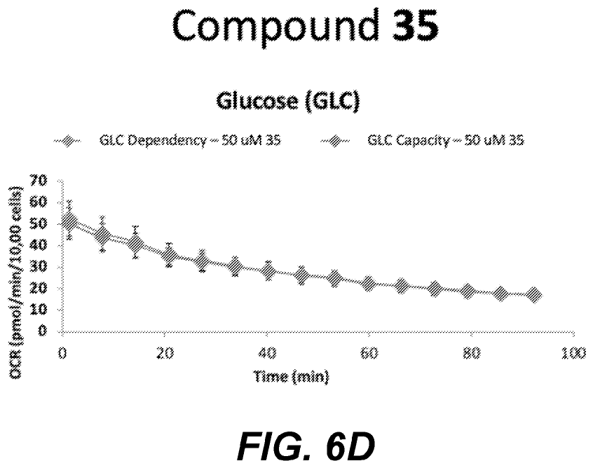

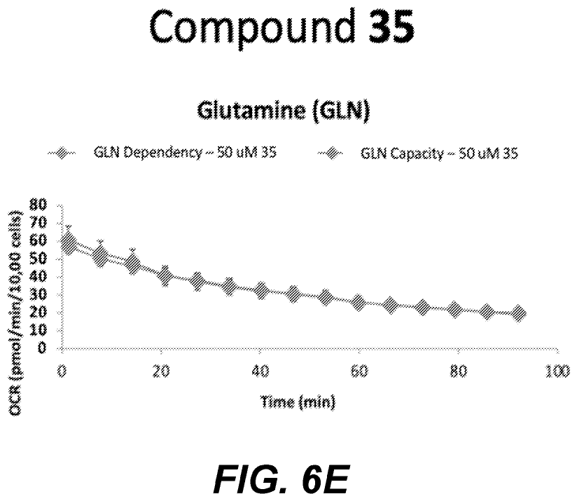

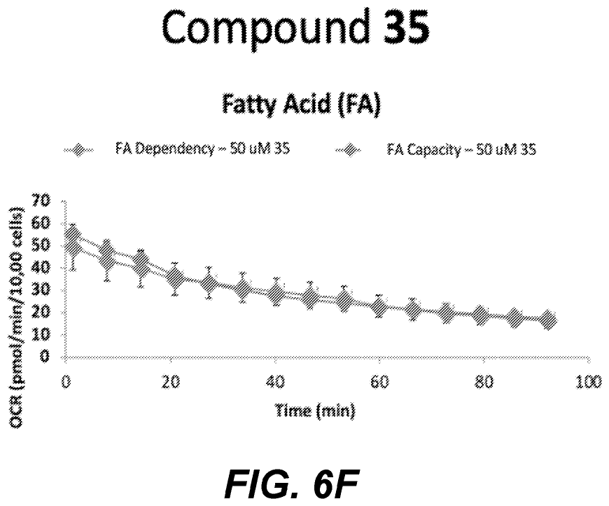

The effects on cellular ATP production via mitochondrial oxidative phosphorylation or glycolysis was quantified (FIG. 5A-C). Injections of oligomycin and rot/AA demonstrated the TPP-containing compounds to decrease ATP production, but tended to produce a Warburg-like decrease in ATP synthesis from oxidative phosphorylation and an increase in ATP from glycolysis. BPTES (glutaminase inhibitor), UK5099 (mitochondrial pyruvate transporter inhibitor), and etomoxir (carnitine palmitoyltransferase-1/CPT-1 inhibitor) were used to compare the effect of 32 versus the TPP-containing molecule, 35, on mitochondrial glutamine, fatty acid, and glucose oxidation following a 6 hr treatment. Compound 32 (50 μM) appeared to shift cells from glucose oxidation toward glutamine metabolism with no effect on fatty acid oxidation (data not shown), which may be an indication of off-target engagement. In contrast, a 6 hr treatment with 35 reduced OCR (oxygen consumption rate) and prevented measurement. Therefore, a 2 hr incubation with 35 was attempted, but the cells were unresponsive to BPTES, UK5099, and etomoxir (FIG. 6A-F), suggesting that all three pathways are disrupted by the inhibitor.

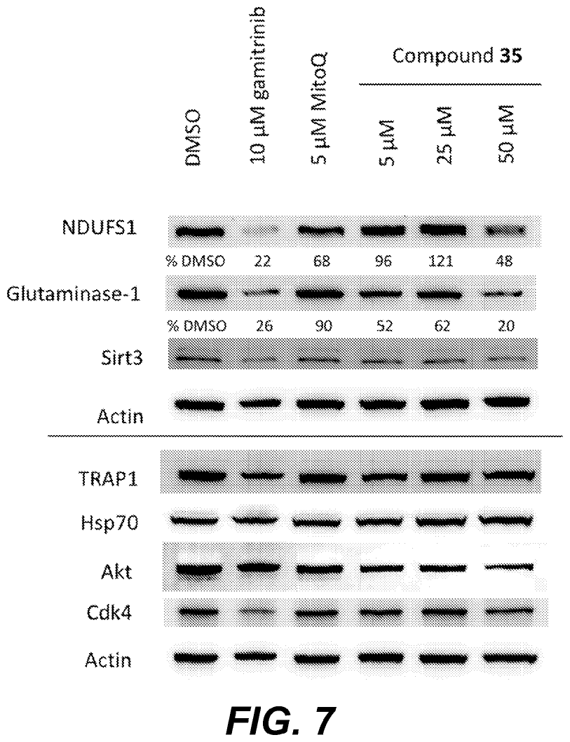

Western blots were then performed to assess whether glutaminase, an enzyme involved in mitochondrial glutamine metabolism, and the other TRAP1 client proteins were degraded at 2 hr. As seen in FIG. 7, the expression of TRAP1 clients NDUFS1, glutaminase-1, and Sirt3 were dose-dependently decreased in response to 50 μM of 35 following a 2 hr treatment. These data provide additional evidence that these enzymes are dependent upon TRAP1 for stabilization.

### Example 5: Structure of TRAP1 Selective Inhibitors Bound to TRAP1's N-Terminal ATP-Binding Site

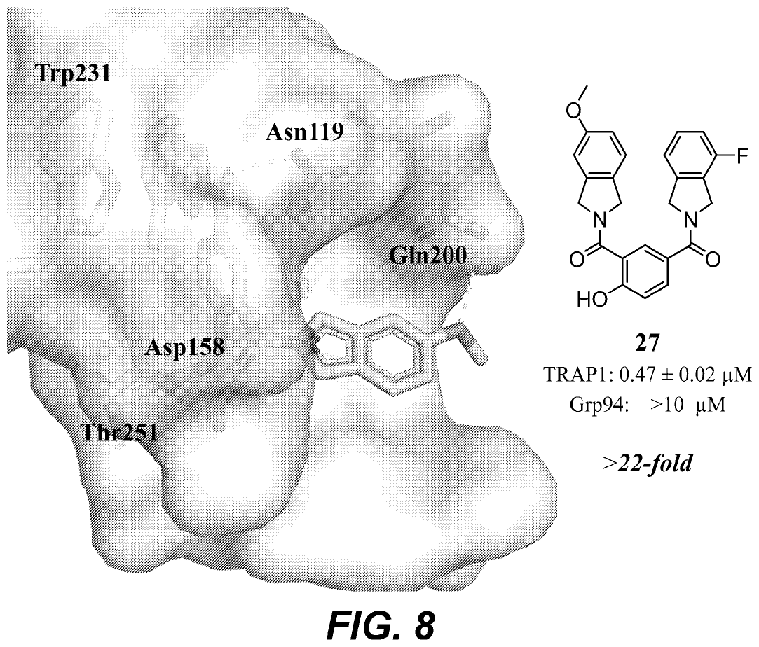

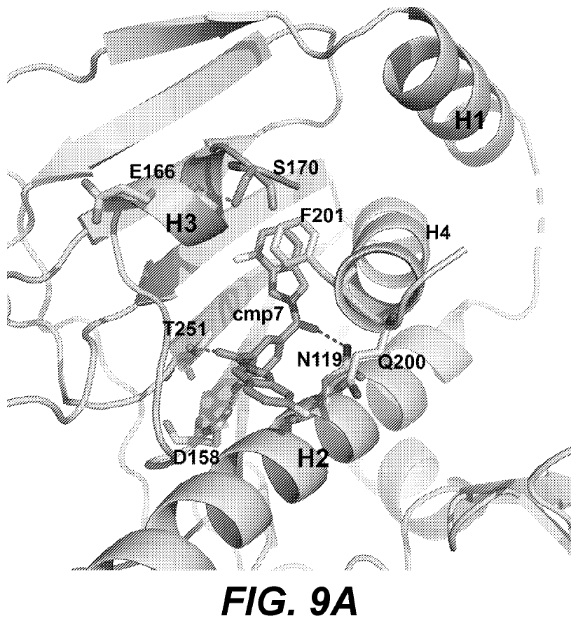

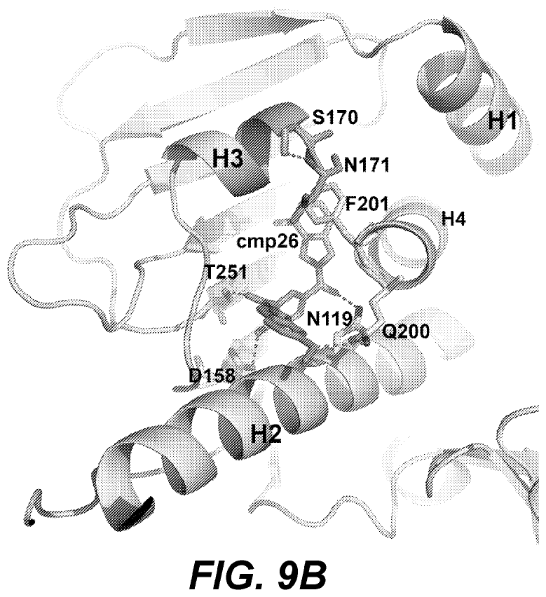

The co-crystal structures of the ATP-binding pocket of TRAP1_NM bound to compounds 7 and 26 were solved and are presented in FIG. 9A-B. The structures of 7 and 26 bound to the TRAP1 N-terminal ATP binding pocket showed R³ and R⁴ to reside at the top of the pocket near Trp231, while the R¹ and R² substitutions were solvent exposed (FIG. 8). As mentioned previously, selective binding to TRAP1 is promoted by the phenol. In fact, removal of the phenol that interacts with the Hsp90a residue Ser52 reduces Hsp90a affinity and tilts the para isoindoline down, allowing the carbonyl to hydrogen bond with Asn119 in TRAP1. As a result, the inhibitor is locked into a conformation that sterically clashes with Phe138 in both cytosolic isoforms, but not in the wider binding pockets that exist within Grp94 and TRAP1.

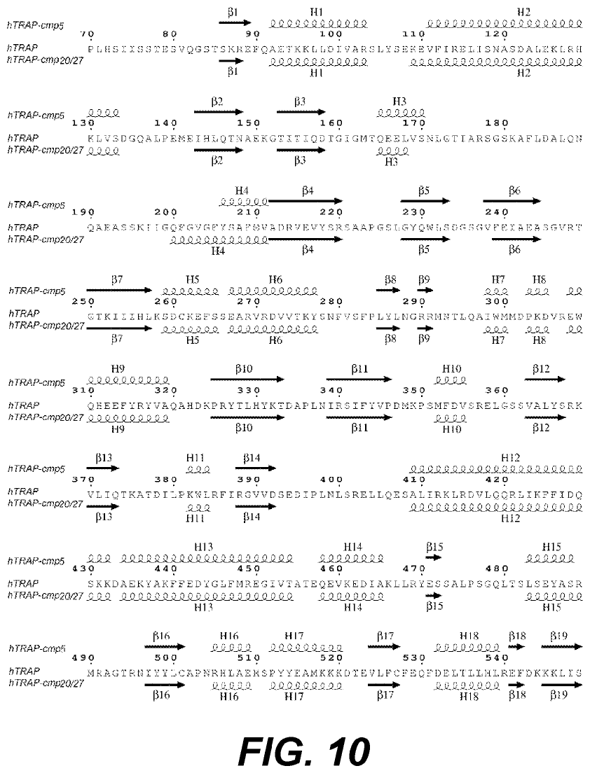

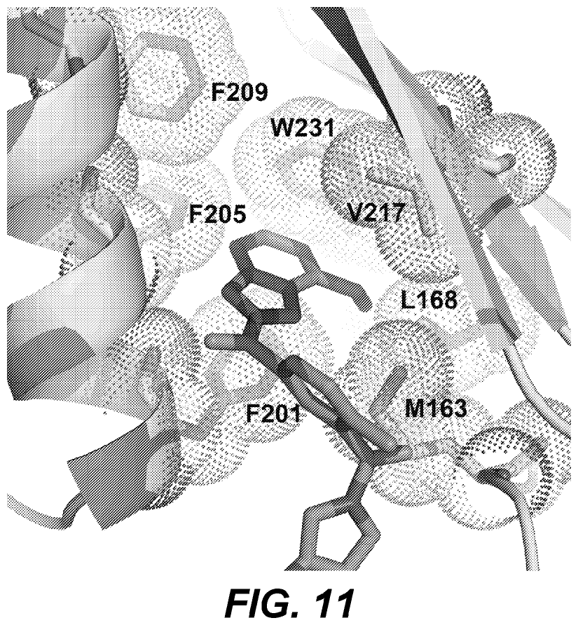

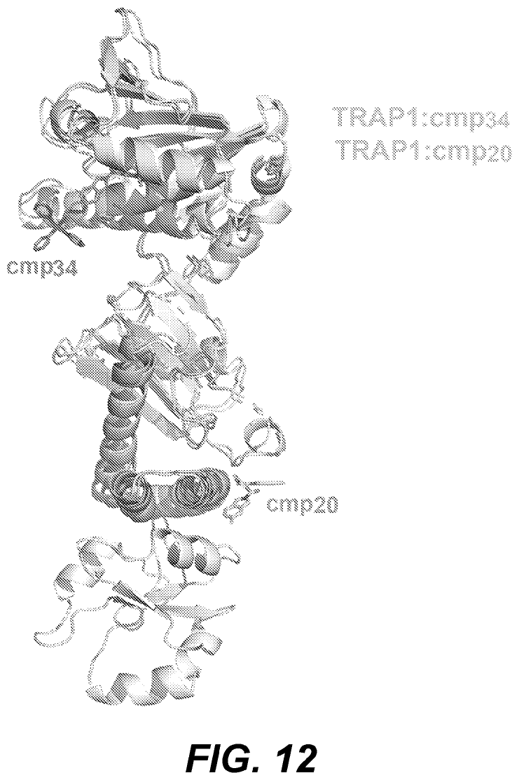

The structures of compounds 20 and 27 bound to TRAP1 provided insight into the ˜10 fold increase in binding affinity for TRAP1 as compared to 5 (FIG. 9A-B). Specifically, the coiled region extending from Asn171 to Ser178 is stabilized by a hydrogen-bond network with water molecules. Gly202 caps the N-terminus of helix 3 (FIG. 10) at Phe205 by shortening the helix one turn and exposing Phe201 to solvent while the backbone carbonyl and amide are stabilized by hydrogen-bonding with water. In the structures of TRAP1_NM bound to 20 and 27, hydrogen-bonding between Gln200 and the electronegative R² substituent extends the helix by one turn, and results in Phe201's ability to π-stack with 20 and 27. Computational studies suggest that increased binding affinity is exhibited by compounds with a methoxy or ethoxy substituent at R⁴ (compounds 25 and 26) is due to deeper penetration into the R⁴ hydrophobic pocket (FIG. 11). The co-crystal structure of 34 bound to the ATP-binding site was similar to the structures of TRAP1-NM bound to 20 and 27. However, the electron density of residues Gln200 and Phe201 were not observed in the co-crystal structure of 34, nor could the amino acids residues on the C-terminal side of Ser472 (15) be assigned (FIG. 12), highlighting the flexible nature of this region. The crystallographic data and statistics for the complex of TRAP1 with compounds 20, 27, 5, and 34 are shown in Table 4.

The N-terminal domain of TRAP1 (TRAP1_N) has been crystallized in a symmetrical coil-coiled apo-conformation (PDB: 5F3K) and co-crystallize with ADPNP (PDB: 5F5R). Overlay of these structures, with co-crystal structures of the inhibitor bound to TRAP1_NM, indicate their conformations are not compatible with the binding of these compounds. While composite models of apo- and ADPNP-bound TRAP1-N built onto full length TRAP1 show they may represent an intermediate conformation compatible with dimer closure, cryo-EM studies clearly show apo-TRAP in an open V-conformation that would be capable of binding the inhibitors reported herein.

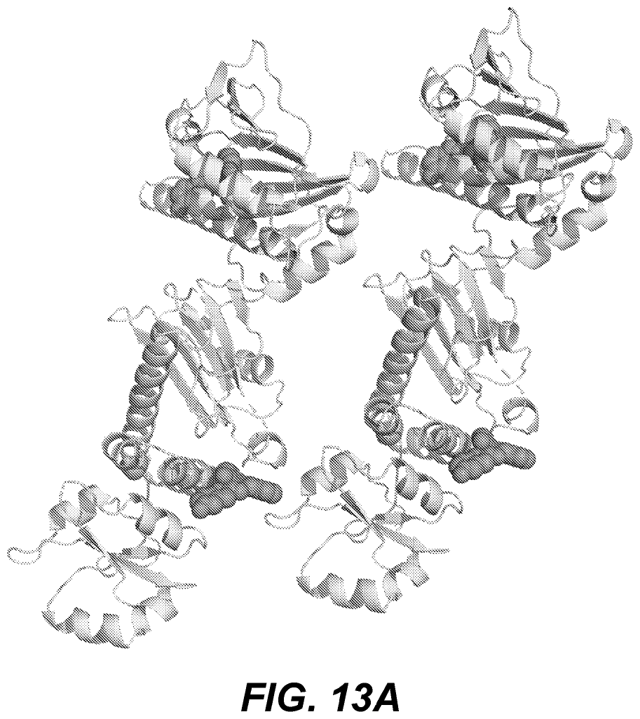

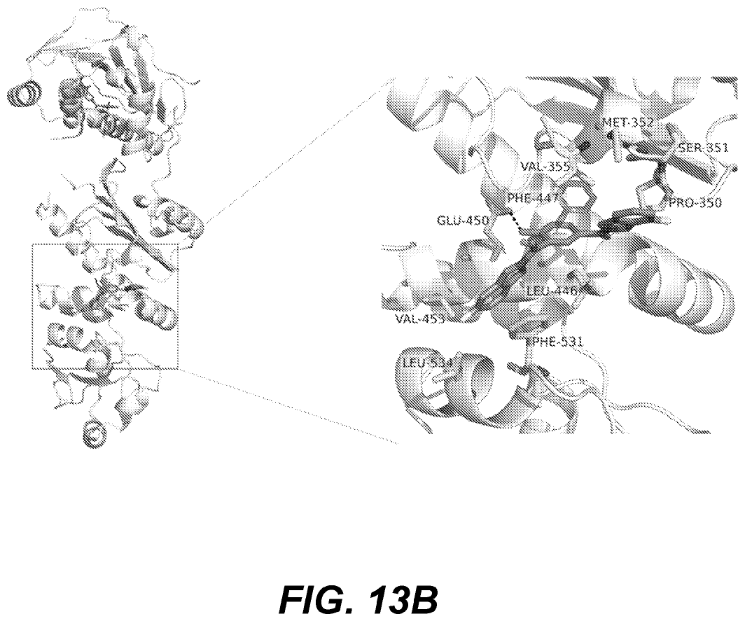

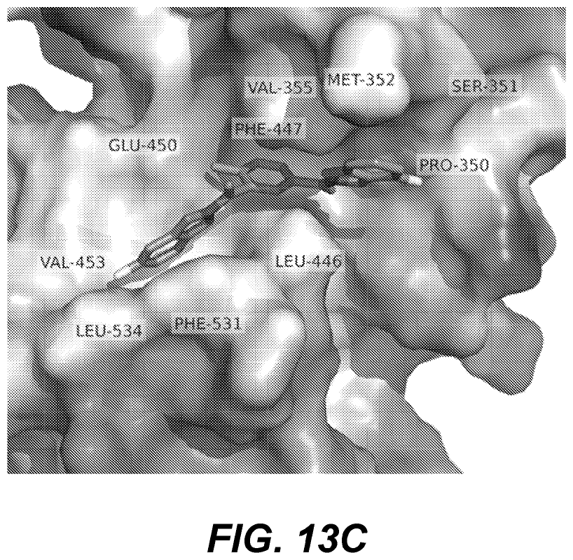

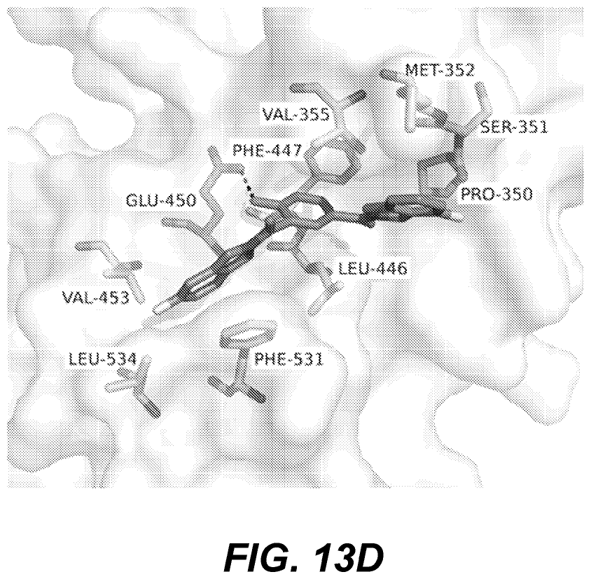

The co-crystal structures of TRAP1_NM bound to 5, 20 and 27 contain two molecules of inhibitor bound to the TRAP1_NM in the symmetrical unit (FIG. 13A). It was noted that the binding site present in one of the TRAP1_NM molecules overlapped with amino acid residues (Pro350, Ser351, Met352, Val355 and Phe447) within an allosteric binding site in the middle domain of zebra fish TRAP1 predicted by Colombo and coworkers. In addition, the bound inhibitors had hydrophobic interactions with Leu446, Val451, Phe531 and Leu534 (FIG. 13B-DC), which were identified as the binding site for MitoQ in zebra fish TRAP1. Glu450 was also observed to form a hydrogen-bond with the hydroxyl moiety of the central benzyl ring. Cryo-EM studies by Agard and coworkers indicate this region of TRAP1 to interact with the folding of its client, succinate dehydrogenase B. This second binding site may represent a target for the development of allosteric modulators of TRAP1.

TABLE 4
Crystallographic data and statistics

| Data collection | hTRAP1-Compound 20 | | hTRAP1-Compound 27 | | hTRAP1-Compound 5 | | hTRAP1-Compound 34 | |
|---|---|---|---|---|---|---|---|---|
| Beamline | 19-ID, APS | | 19-ID, APS | | 19ID, APS | | 19-ID, APS | |
| Wavelength, Å | 0.97935 | | 0.97935 | | 0.97918 | | 0.97951 | |
| Space group | P2₁2₁2₁ | | P2₁2₁2₁ | | P2₁2₁2₁ | | P2₁ | |
| Cell parameters a, b, c, Å | 42.1, 68.9, 182.5 | | 41.8, 68.9. 183.1 | | 42.3, 68.7, 182.6 | | 66.37, 118.59, 69.39; | |
| | | | | | | | β = 90.87° | |
| Resolution, Å | 50.00-1.70 | (1.76-1.70) | 50.00-1.60 | (1.66-1.60) | 50.00-1.45 | (1.50-1.45) | 50.00-3.10 | (3.21-3.10) |
| Total reflections | 348,167 | | 409,892 | | 596,128 | | 117,332 | |
| Unique reflections | 58,263 | (5,614) | 70.222 | (6,727) | 95,177 | (9,055) | 19,398 | (1,910) |
| Redundancy | 6.0 | (4.6) | 5.8 | (4.9) | 6.3 | (5.3) | 6.0 | (4.6) |
| Completeness, % | 99.7 | (97.9) | 99.3 | (97.2) | 99.4 | (96.0) | 98.6 | (96.0) |
| I/σ | 25.6 | (3.5) | 24.0 | (1.6) | 31.8 | (2.4) | 11.2 | (1.11) |
| R [symbol] | 8.3 | (37.6) | 10.1 | (90.8) | 6.5 | (56.1) | 23.8 | (156.9) |
| Refinement statistics | | | | | | | | |
| Resolution range used. Å | 38.21-1.71 | (1.75-1.71) | 45.79-1.60 | (1.64-1.60) | 35.33-1.45 | (1.48-1.45) | 48.32-3.10 | (3.15-3.10) |
| No. reflections used | 57,082 | (3,162) | 70,085 | (4,508) | 95,078 | (6,028) | 18,185 | |
| R [symbol] /R [symbol] | 18.20/20.37 | | 18.94/21.25 | | 18.09/20.20 | | 22.0/31.1 | |
| Rmsd bond lengths, Å | 0.007 | | 0.006 | | 0.005 | | 0.010 | |
| Rmsd bond angles, ° | 0.888 | | 0.897 | | 0.837 | | 1.542 | |
| Number of atoms (average B, Å²) | | | | | | | | |
| Protein | 3.585 | (37.7) | 3,568 | (40.5) | 3,669 | (30.2) | 6,737 | (101.6) |
| Ligand | 62 | (33.3) | 64 | (39.2) | 58 | (24.5) | 104 | (105.5) |
| Water | 327 | (43.8) | 316 | (46.3) | 539 | (40.9) | 0 | |
| Ramachandran values | | | | | | | | |

| | | | | |
|---|---|---|---|---|
| Preferred regions, % | 97.2 | 98.6 | 98.4 | 91.4 |
| Allowed regions, % | 2.8 | 1.4 | 1.6 | 8.6 |

Values in parentheses are for the highest-resolution shell.
R [symbol] = Σ |I [symbol] − I [symbol] |/Σ I [symbol] , R [symbol] = Σ|| F_{obs} | − |F_{calc}||/Σ Fobs [symbol] R [symbol] was calculated using 5% of data.|

[symbol]
indicates data missing or illegible when filed

### CONCLUSIONS

A structure-based approach toward the development of an N-terminal, TRAP1-selective inhibitor led to the development of a selective and potent TRAP1 inhibitor that was used to interrogate TRAP1's biological activity. Molecular modeling and modification of the hit compound (5) led to an asymmetric phenol that exhibited 13-fold selectivity over Grp94, the Hsp90 paralog most similar to TRAP1. SAR investigation and the solution of four co-crystal structures provided the foundation for a rational drug design approach, which ultimately yielded a 40 nM inhibitor that manifests >250-fold TRAP1 selectivity versus Grp94. The introduction of dimethylamine or triphenylphosphonium moieties did not affect TRAP1 affinity, but the TPP moiety was required for the molecules to engage TRAP1 in the mitochondria in HeLa and 22Rv1 cells. These newly synthesized molecules with the mitochondrial-targeting TPP moiety selectively induced TRAP1 client protein degradation without inducing the heat shock response. Compounds 35 and 36 were shown to inhibit OXPHOS, alter cellular metabolism towards glycolysis, and disrupt the mitochondrial membrane potential. Subsequent optimization of such compounds is likely to result in new therapeutic opportunities that are dependent upon these mitochondrial processes.

The breadth and scope of the present disclosure should not be limited by any of the above-described exemplary aspects but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the disclosure are set out in the following numbered clauses:
Clause 1. A compound of formula (I), or a pharmaceutically acceptable salt thereof,

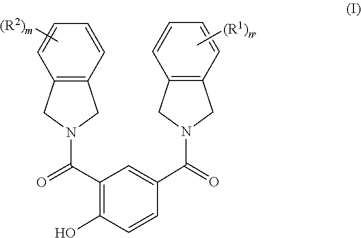

wherein:
R¹, at each occurrence, is independently C₁₋₆alkyl, C₁₋₄haloalkyl, halogen, cyano, —N(R^{1a})₂, —OR^{1b}, —SR^{1b}, —C(O)R^{1b}, —CO₂R^{1b}, —C(O)N(R^{1a})₂, —SO₂R^{1a}, -L¹-Y¹, —O-L¹-Y¹, —S-L¹-Y¹, —N(R^{1a})-L¹-Y¹, G¹, or —OG¹;
R^{1a}, at each occurrence, is independently hydrogen, C₁₋₄alkyl, or —C(O)C₁₋₄alkyl;
R^{1b}, at each occurrence, is independently hydrogen, C₁₋₄alkyl, C₁₋₂haloalkyl, or —C(O)C₁₋₄alkyl;
L¹, at each occurrence, is independently C₁₋₆alkylene, C₂₋₆alkenylene, or C₂₋₆alkynylene;
Y¹, at each occurrence, is independently hydrogen, cyano, halogen, haloalkyl, —OH, —N(R^{1a})₂, —OR^{1b}, —SR^{1b}, —C(O)R^{1b}, —CO₂R^{1b}, —C(O)N(R^{1a})₂, —SO₂R^{1a},

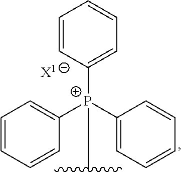

G¹, or —OG¹;
X¹ ⊕ is a pharmaceutically acceptable anion;
G¹ is C₃₋₆cycloalkyl or phenyl, wherein G¹ is optionally substituted with 1-4 substituents selected from the group consisting of C₁₋₄alkyl, C₁₋₂haloalkyl, halogen, cyano, —OC₁₋₄alkyl, and —OC₁₋₂haloalkyl;
R², at each occurrence, is independently C₁₋₆alkyl, C₁₋₄haloalkyl, halogen, cyano, —N(R^{2a})₂, —OR^{2b}, —SR^{2b}, —C(O)R^{2b}, —CO₂R^{2b}, —C(O)N(R^{2a})₂, —SO₂R^{2a}, -L²-Y², —O-L²-Y², —S-L²-Y², —N(R^{2a})-L²_Y², G², or —OG²;
R^{2a}, at each occurrence, is independently hydrogen, C₁₋₄alkyl, or —C(O)C₁₋₄alkyl;
R^{2b}, at each occurrence, is independently hydrogen, C₁₋₄alkyl, C₁₋₂haloalkyl, or —C(O)C₁₋₄alkyl;
L², at each occurrence, is independently C₁₋₆alkylene, C₂₋₆alkenylene, or C₂₋₆alkynylene;
Y², at each occurrence, is independently hydrogen, cyano, halogen, haloalkyl, —OH, —N(R^{2a})₂, —OR^{2b}, —SR^{2b}, —C(O)R^{2b}, —CO₂R^{2b}, —C(O)N(R^{2a})₂, —SO₂R^{2a},

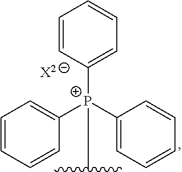

G², or —OG²;
X² ⊕ is a pharmaceutically acceptable anion;
G² is C₃₋₆cycloalkyl or phenyl, wherein G² is optionally substituted with 1-4 substituents selected from the group consisting of C₁₋₄alkyl, C₁₋₂haloalkyl, halogen, cyano, —OC₁₋₄alkyl, and —OC₁₋₂haloalkyl;
n is 0, 1, 2, 3, or 4; and
m is 0, 1, 2, 3, or 4.
Clause 2. The compound of clause 1, or a pharmaceutically acceptable salt thereof, wherein R¹ is halogen, C₁₋₆alkyl, or —OR^{1b}.
Clause 3. The compound of clause 1 or 2, or a pharmaceutically acceptable salt thereof, wherein R^{1b} is C₁₋₄alkyl.
Clause 4. The compound of any one of clauses 1-3, or a pharmaceutically acceptable salt thereof, wherein R¹ is fluoro, chloro, bromo, —CH₃, —OCH₃, or —OC₂H₅.
Clause 5. The compound of any one of clauses 1-4, or a pharmaceutically acceptable salt thereof, wherein R² is halogen, —OR^{2b}, or —O-L²-Y².
Clause 6. The compound of any one of clauses 1-5, or a pharmaceutically acceptable salt thereof, wherein R^{2b} is C₁₋₄alkyl.
Clause 7. The compound of any one of clauses 1-6, or a pharmaceutically acceptable salt thereof, wherein L² is C₁₋₆alkylene.
Clause 8. The compound of any one of clauses 1-7, or a pharmaceutically acceptable salt thereof, wherein Y² is —N(R^{2a})₂ or

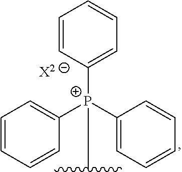

Clause 9. The compound of any one of clauses 1-8, or a pharmaceutically acceptable salt thereof, wherein R^{2a} is C₁₋₄alkyl.
Clause 10. The compound of any one of clauses 1-9, wherein n is 0, 1, or 2.
Clause 11. The compound of any one of clauses 1-10, wherein m is 0, 1, or 2.
Clause 12. The compound of any one of clauses 1-11, or a pharmaceutically acceptable salt thereof, wherein the compound of formula (I) is a compound of formula (I-a):

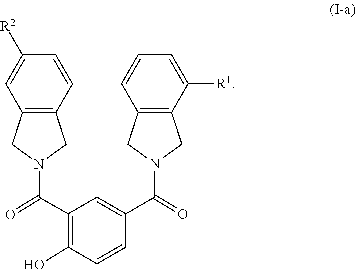

Clause 13. The compound of any one of clauses 1-12, or a pharmaceutically acceptable salt thereof, wherein the compound of formula (I) is a compound of formula (I-b):

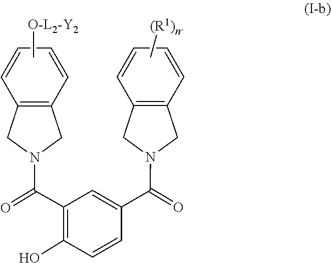

Clause 14. The compound of any one of clauses 1-13, or a pharmaceutically acceptable salt thereof, wherein the compound of formula (I) is a compound of formula (1-ab):

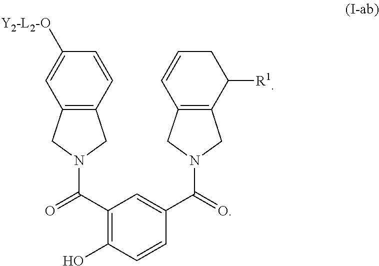

Clause 15. The compound of any one of clauses 1-14, wherein the compound of formula (I) is selected from the group consisting of:

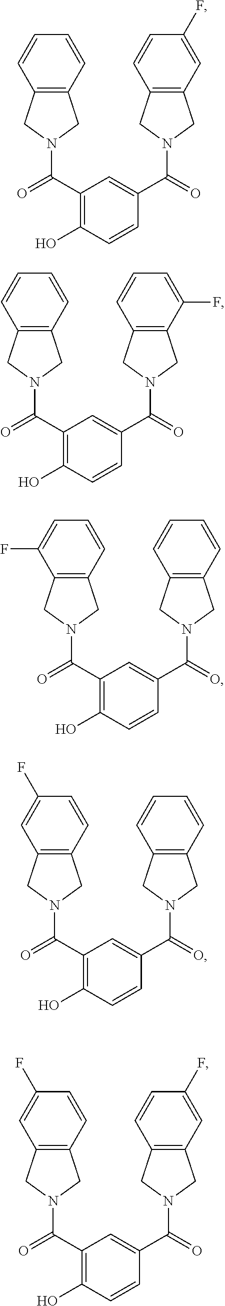

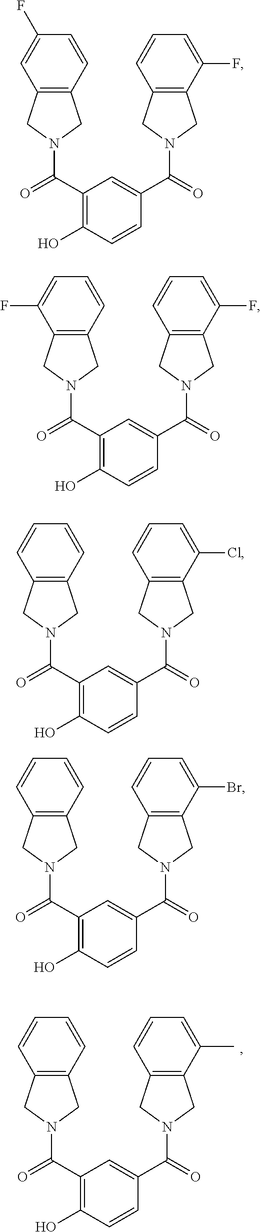

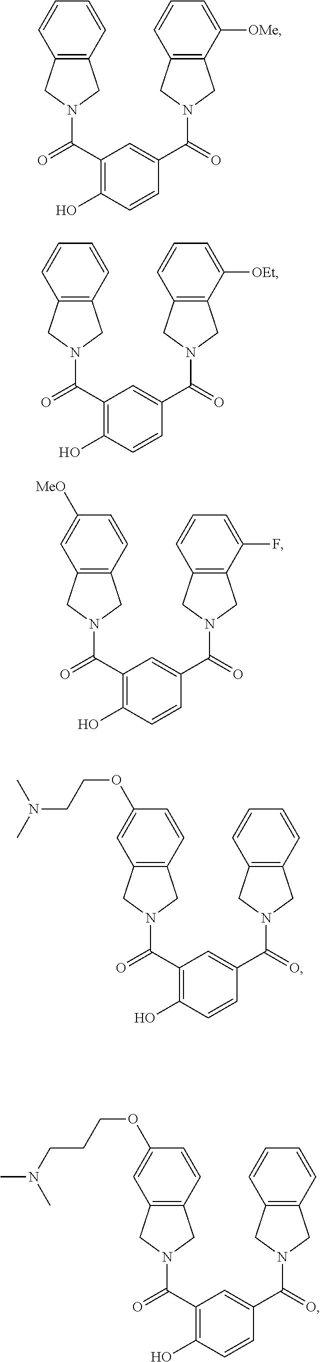

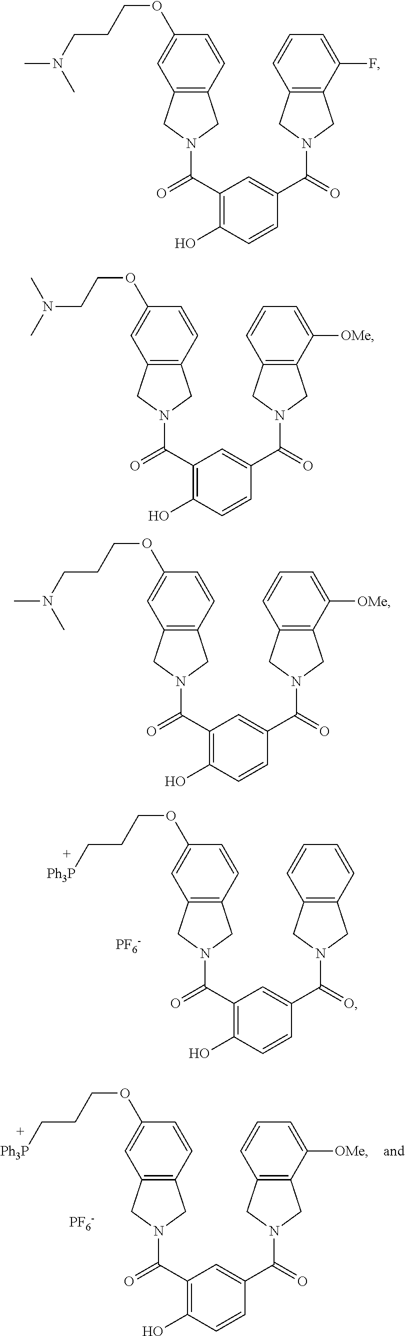

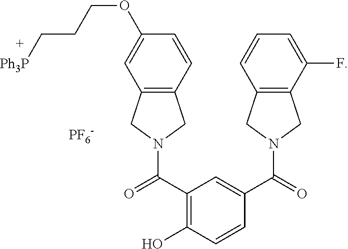

Clause 16. A pharmaceutical composition comprising the compound of any one of clauses 1-15, or a pharmaceutically acceptable salt thereof, and a pharmaceutically acceptable carrier.
Clause 17. A method for treating a disease or disorder associated with Tumor Necrosis Factor Receptor-Associated Protein 1 (TRAP-1) dysfunction comprising administering to a subject in need thereof, a therapeutically effective amount of the compound of any one of clauses 1-15, or a pharmaceutically acceptable salt thereof, or the pharmaceutical composition of clause 16.
Clause 18. The method of clause 17, wherein the disease or disorder is cancer.
Clause 19. A compound of any one of clauses 1-15, or a pharmaceutically acceptable salt thereof, or the pharmaceutical composition of clause 16, for use in the treatment of a disease or disorder associated with Tumor Necrosis Factor Receptor-Associated Protein 1 (TRAP-1) dysfunction.
Clause 20. Use of a compound of any one of clauses 1-15, or a pharmaceutically acceptable salt thereof, or the pharmaceutical composition of claim 16, in the manufacture of a medicament for the treatment of a disease or disorder.

1. A compound of formula (I), or a pharmaceutically acceptable salt thereof, 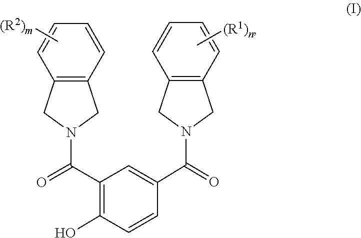
wherein:
R¹, at each occurrence, is independently C₁₋₆alkyl, C₁₋₄haloalkyl, halogen, cyano, —N(R^{1a})₂—OR^{1b}, —SR^{1b}, —C(O)R^{1b}, —CO₂R^{1b}, —C(O)N(R^{1a})₂, —SO₂R^{1a}, -L¹-Y¹, —O-L¹-Y¹, —S-L¹-Y¹, —N(R^{1a})-L¹-Y¹, G¹, or —OG¹;
R^{1a}, at each occurrence, is independently hydrogen, C₁₋₄alkyl, or —C(O)C₁₋₄alkyl;
R^{1b}, at each occurrence, is independently hydrogen, C₁₋₄alkyl, C₁₋₂haloalkyl, or —C(O)C₁₋₄alkyl;
L¹, at each occurrence, is independently C₁₋₆alkylene, C₂₋₆alkenylene, or C₂₋₆alkynylene;
Y¹, at each occurrence, is independently hydrogen, cyano, halogen, haloalkyl, —OH, —N(R^{1a})₂, —OR^{1b}, —SR^{1b}, —C(O)R^{1b}, —CO₂R^{1b}, —C(O)N(R^{1a})₂, —SO₂R^{1a}, 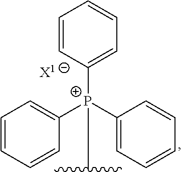
G¹, or —OG¹;
X¹ ⊕ is a pharmaceutically acceptable anion;
G¹ is C₃₋₆cycloalkyl or phenyl, wherein G¹ is optionally substituted with 1-4 substituents selected from the group consisting of C₁₋₄alkyl, C₁₋₂haloalkyl, halogen, cyano, —OC₁₋₄alkyl, and —OC₁₋₂haloalkyl;
R², at each occurrence, is independently C₁₋₆alkyl, C₁₋₄haloalkyl, halogen, cyano, —N(R^{2a})₂, —OR^{2b}, —SR^{2b}, —C(O)R^{2b}, —CO₂R^{2b}, —C(O)N(R^{2a})₂, —SO₂R^{2a}, -L²-Y², —O-L²-Y², —S-L²-Y², —N(R^{2a})-L²-Y², G², or —OG²;
R^{2a}, at each occurrence, is independently hydrogen, C₁₋₄alkyl, or —C(O)C₁₋₄alkyl;
R^{2b}, at each occurrence, is independently hydrogen, C₁₋₄alkyl, C₁₋₂haloalkyl, or —C(O)C₁₋₄alkyl;
L², at each occurrence, is independently C₁₋₆alkylene, C₂₋₆alkenylene, or C₂₋₆alkynylene;
Y² at each occurrence, is independently hydrogen, cyano, halogen, haloalkyl, —OH, —N(R^{2a})₂, —OR^{2b}, —SR^{2b}, —C(O)R^{2b}, —CO₂R^{2b}, —C(O)N(R^{2a})₂, —SO₂R^{2a}, 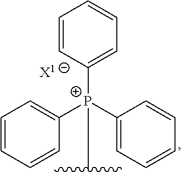
G², or OG²;
X² ⊕ is a pharmaceutically acceptable anion;
G² is C₃₋₆cycloalkyl or phenyl, wherein G² is optionally substituted with 1-4 substituents selected from the group consisting of C₁₋₄alkyl, C₁₋₂haloalkyl, halogen, cyano, —OC₁₋₄alkyl, and —OC₁₋₂haloalkyl;
n is 0, 1, 2, 3, or 4; and
n is 0, 1, 2, 3, or 4.

2. The compound of claim 1, or a pharmaceutically acceptable salt thereof, wherein R¹ is halogen, C₁₋₆alkyl, or —OR^{1b}.

3. The compound of claim 1, or a pharmaceutically acceptable salt thereof, wherein R^{1b} is C₁₋₄alkyl.

4. The compound of claim 1, or a pharmaceutically acceptable salt thereof, wherein R¹ is fluoro, chloro, bromo, —CH₃, —OCH₃, or —OC₂H₅.

5. The compound of claim 1, or a pharmaceutically acceptable salt thereof, wherein R² is halogen, —OR^{2b}, or —O-L²-Y².

6. The compound of claim 1, or a pharmaceutically acceptable salt thereof, wherein R^{2b} is C₁₋₄alkyl.

7. The compound of claim 1, or a pharmaceutically acceptable salt thereof, wherein L² is C₁₋₆alkylene.

8. The compound of claim 1, or a pharmaceutically acceptable salt thereof, wherein Y² is —N(R^{2a})₂ or

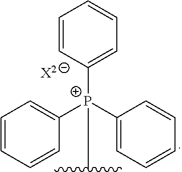

9. The compound of claim 1, or a pharmaceutically acceptable salt thereof, wherein R^{2a} is C₁₋₄alkyl.

10. The compound of claim 1, wherein n is 0, 1, or 2.

11. The compound of claim 1, wherein m is 0, 1, or 2.

12. The compound of claim 1, or a pharmaceutically acceptable salt thereof, wherein the compound of formula (I) is a compound of formula (I-a):

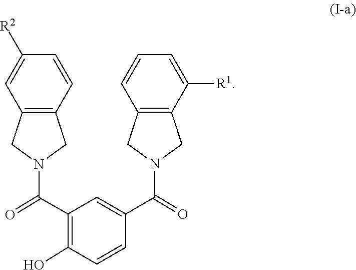

13. The compound of claim 1, or a pharmaceutically acceptable salt thereof, wherein the compound of formula (I) is a compound of formula (I-b):

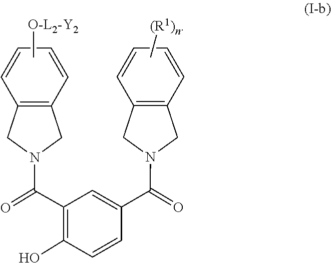

14. The compound of claim 1, or a pharmaceutically acceptable salt thereof, wherein the compound of formula (I) is a compound of formula (1-ab):

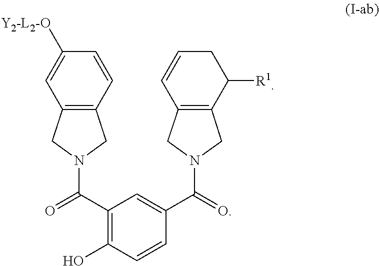

15. The compound of claim 1, wherein the compound of formula (I) is selected from the group consisting of:

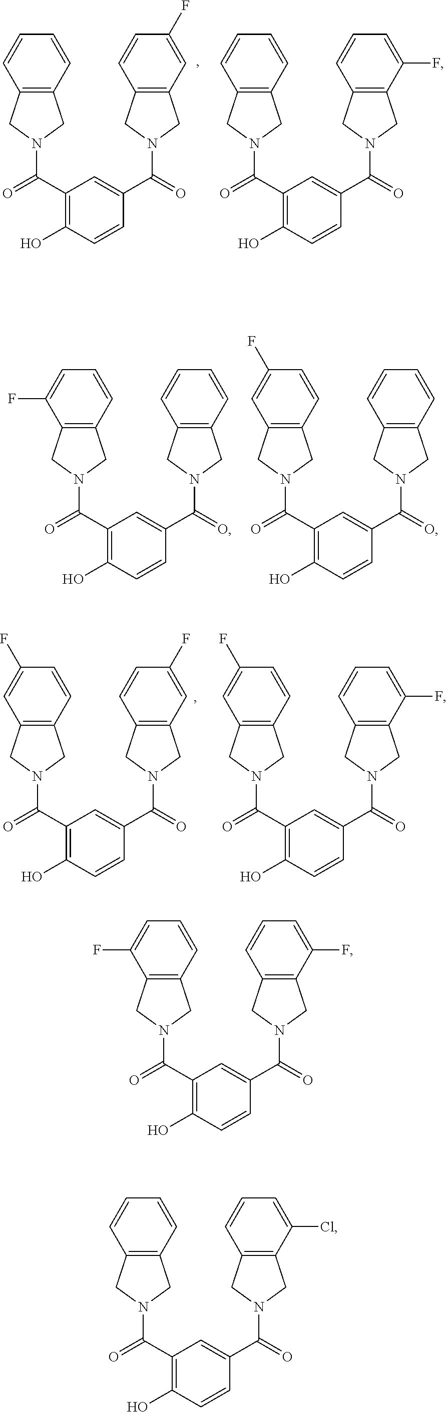

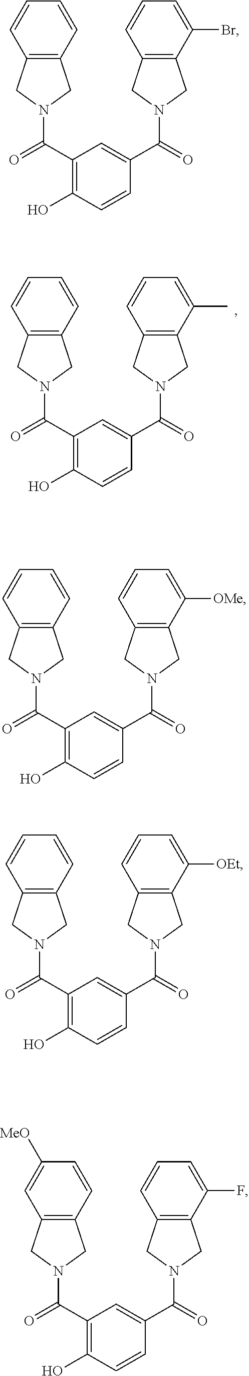 [SMILES: O=C(c1ccc(O)c(C(=O)N2Cc3ccccc3C2)c1)N1Cc2cccc(Br)c2C1 ; Cc1cccc2c1CN(C(=O)c1ccc(O)c(C(=O)N3Cc4ccccc4C3)c1)C2 ; COc1cccc2c1CN(C(=O)c1ccc(O)c(C(=O)N3Cc4ccccc4C3)c1)C2 ; CCOc1cccc2c1CN(C(=O)c1ccc(O)c(C(=O)N3Cc4ccccc4C3)c1)C2 ; COc1ccc2c(c1)CN(C(=O)c1cc(C(=O)N3Cc4cccc(F)c4C3)ccc1O)C2]

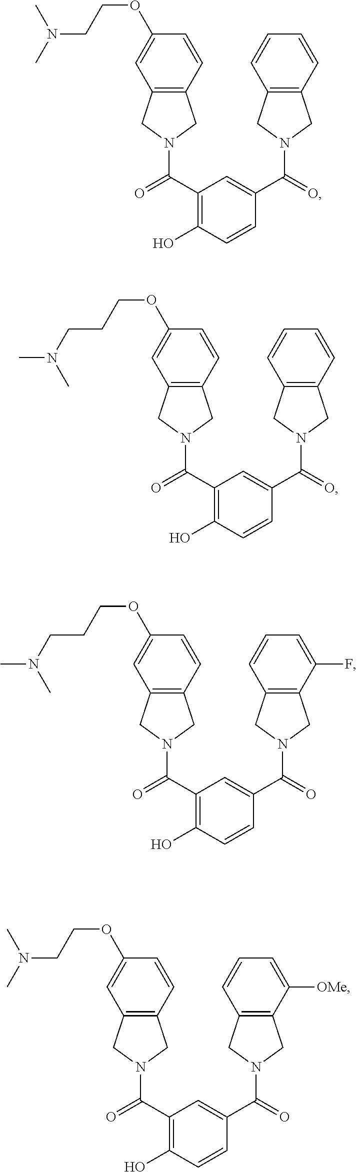 [SMILES: CN(C)CCOc1ccc2c(c1)CN(C(=O)c1cc(C(=O)N3Cc4ccccc4C3)ccc1O)C2 ; CN(C)CCCOc1ccc2c(c1)CN(C(=O)c1cc(C(=O)N3Cc4ccccc4C3)ccc1O)C2 ; CN(C)CCCOc1ccc2c(c1)CN(C(=O)c1cc(C(=O)N3Cc4cccc(F)c4C3)ccc1O)C2 ; COc1cccc2c1CN(C(=O)c1ccc(O)c(C(=O)N3Cc4ccc(OCCN(C)C)cc4C3)c1)C2]

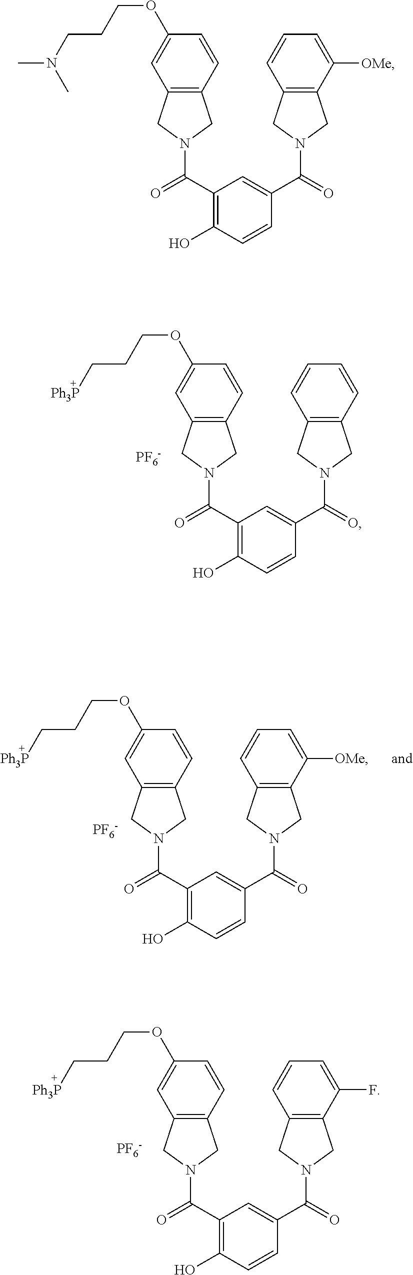

16. A pharmaceutical composition comprising the compound of claim 1, or a pharmaceutically acceptable salt thereof, and a pharmaceutically acceptable carrier.

17. A method for treating a disease or disorder associated with Tumor Necrosis Factor Receptor-Associated Protein 1 (TRAP-1) dysfunction comprising administering to a subject in need thereof, a therapeutically effective amount of the compound of claim 1, or a pharmaceutically acceptable salt thereof, or the pharmaceutical composition of claim 16.

18. The method of claim 17, wherein the disease or disorder is cancer.

19. A compound of claim 1, or a pharmaceutically acceptable salt thereof, or the pharmaceutical composition of claim 16, for use in the treatment of a disease or disorder associated with Tumor Necrosis Factor Receptor-Associated Protein 1 (TRAP-1) dysfunction.

20. Use of a compound of claim 1, or a pharmaceutically acceptable salt thereof, or the pharmaceutical composition of claim 16, in the manufacture of a medicament for the treatment of a disease or disorder.
